(19) United States
(12) Patent Application Publication
(10) Pub. No.: US 2015/0335657 A1
(43) Pub. Date: Nov. 26, 2015

(54) Methods for Modulating Sirtuin Enzymes

(71) Applicant: The Trustees of Princeton University, Princeton, NJ (US)

(72) Inventors: Thomas Shenk, Princeton, NJ (US); Emre Koyuncu, Princeton, NJ (US); Hahn Kim, Princeton, NJ (US); Ileana Cristea, Princeton, NJ (US); David MacMillan, Princeton, NJ (US)

(21) Appl. No.: 14/703,371

(22) Filed: May 4, 2015

Related U.S. Application Data
(60) Provisional application No. 61/988,682, filed on May 5, 2014.

(51) Int. Cl. A61K 31/554; A61K 31/519; A61K 45/06; A61K 31/502; A61K 31/4709; A61K 31/4155; A61K 31/444; A61K 31/4439; A61K 31/42; A61K 31/4178; A61K 31/4164; A61K 31/553; A61K 31/4412; A61K 31/496; A61K 31/4015; A61K 31/415; A61K 31/4725; A61K 31/501; A61K 31/47

(52) U.S. Cl. CPC A61K 31/554; A61K 31/47; A61K 31/519; A61K 45/06; A61K 31/502; A61K 31/4709; A61K 31/4155; A61K 31/444; A61K 31/4439; A61K 31/42; A61K 31/4178; A61K 31/4164; A61K 31/553; A61K 31/4412; A61K 31/496; A61K 31/4015; A61K 31/415; A61K 31/4725; A61K 31/501

(57) ABSTRACT

This document relates to compounds useful for modulating sirtuin enzymes. For example, the compounds provided herein are useful as broad spectrum antiviral agents. In addition, the compounds provided herein may be used in the treatment of other disorders associated with sirtuin enzymes, such as diseases related to aging and stress, blood clotting disorders, cancer, cardiovascular diseases, diabetes, inflammation, neurodegenerative diseases, and obesity.

### CROSS-REFERENCE TO RELATED APPLICATIONS

This application claims the benefit of U.S. Provisional Application No. 61/988,682, filed May 5, 2014, which is incorporated herein by reference in its entirety.

### TECHNICAL FIELD

This document relates to compounds useful for modulating sirtuin enzymes. For example, the compounds provided herein are useful as broad spectrum antiviral agents.

### BACKGROUND

Acetylation and deacetylation of histone proteins, transcription factors, and related proteins are thought to play a key role in the control of cellular processes. For example, the acetylation state of histones controls how tightly the histone proteins interact with DNA5 and therefore how accessible the DNA is to transcription factors. Enzymes that add acetyl groups to histones or other proteins are called histone acetyltransferases (HATs). Enzymes that remove the acetyl groups fall into two families: the histone deacetylases (HDACs) and the Silent Information Regulator (SIR, SIRT or sirtuin) family of deacetylases. Currently, there are eleven known members of the mammalian HDAC family and seven members of the SIR family (i.e. SIRT1, SIRT2, SIRT3, SIRT4, SIRT5, SIRT6, and SIRT7). The SIR family of genes represents a highly conserved group of genes present in the genomes of organisms ranging from archaebacteria to a variety of eukaryotes. The encoded SIR proteins are involved in diverse processes from regulation of gene silencing to DNA repair.

### SUMMARY

Provided herein are compounds useful for modulating sirtuin enzymes. Compounds provided herein include:

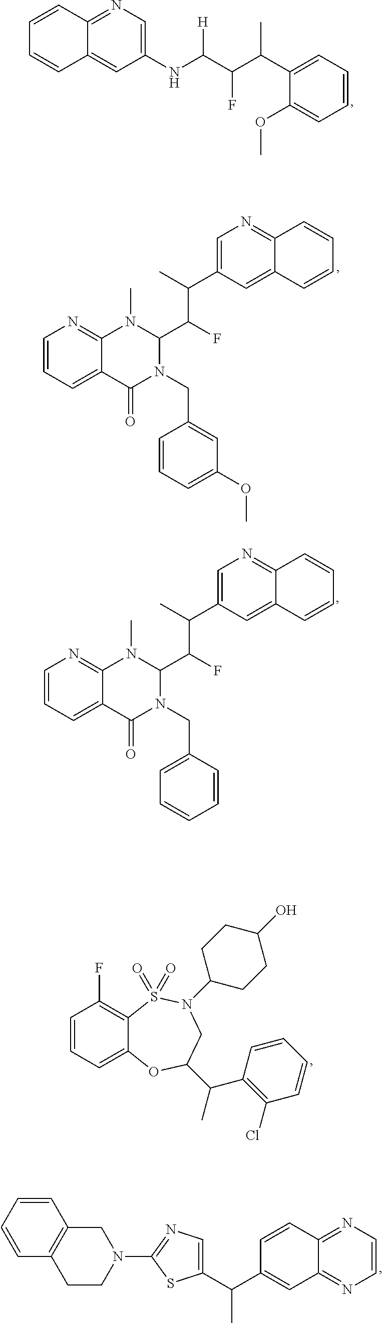

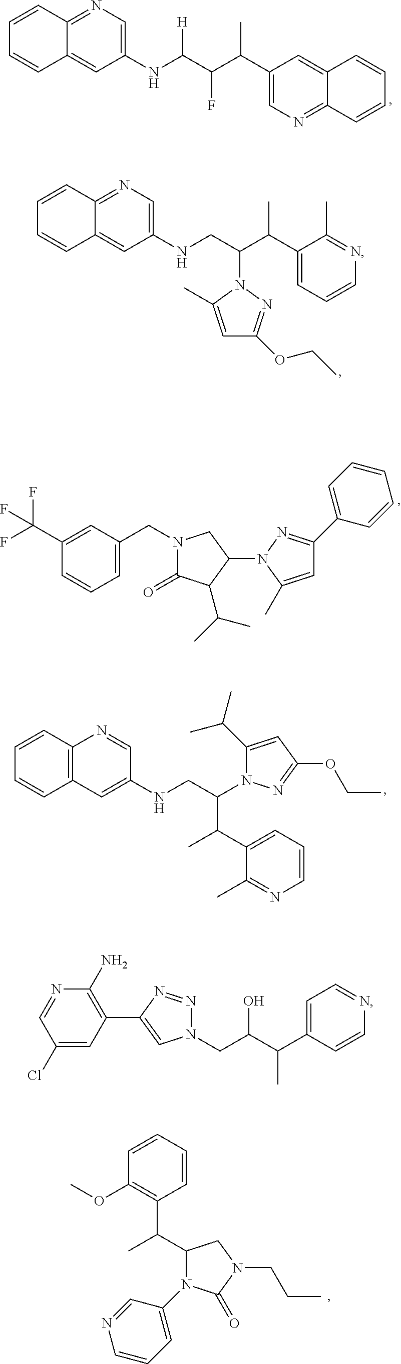

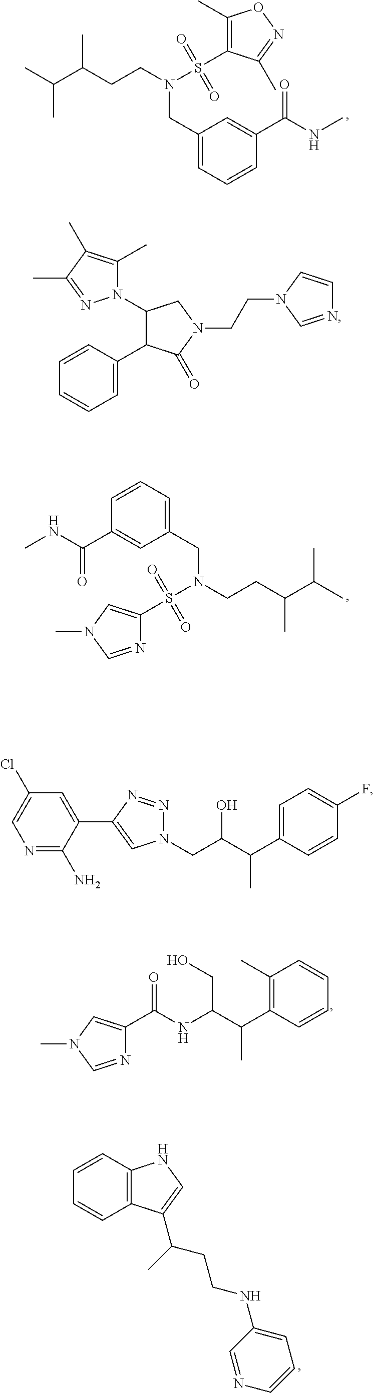

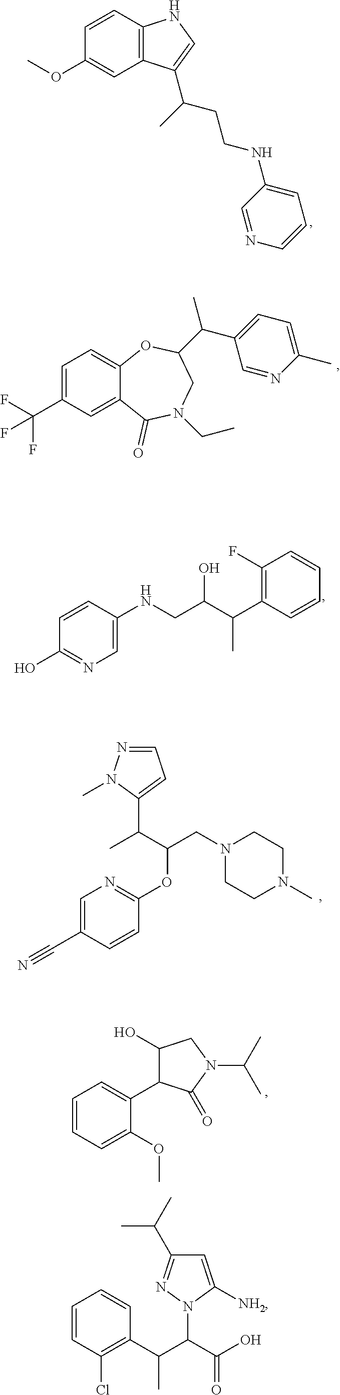

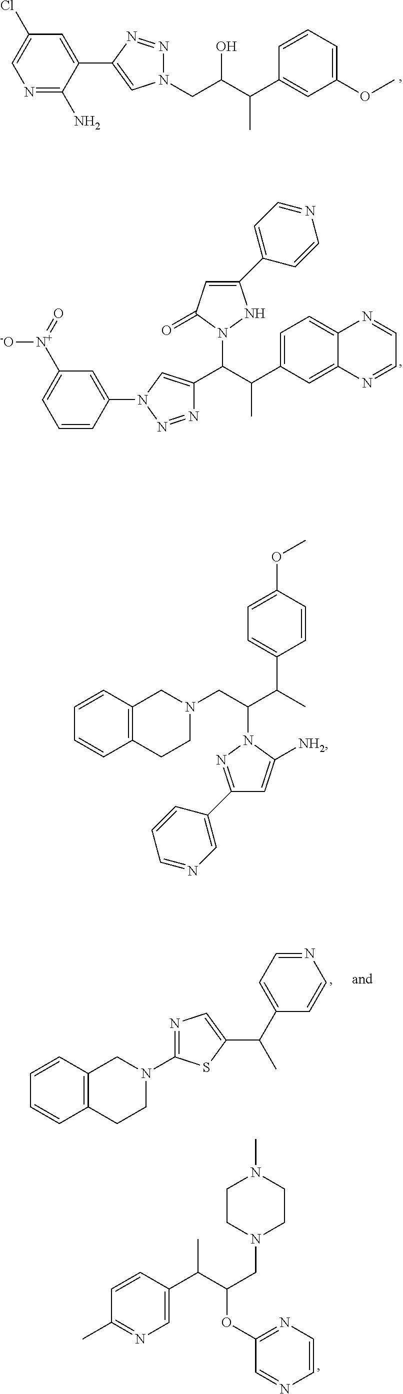

or a pharmaceutically acceptable salt form thereof. The compounds provided herein can be used to treat or prevent a viral infection in a subject, such methods can include administering to the subject a therapeutically effective amount of a compound provided herein, or a pharmaceutically acceptable salt thereof.

In some embodiments, a compound provided herein can inhibit virus production. The method can include contacting a virus-infected cell with a therapeutically effective amount of a compound provided herein, or a pharmaceutically acceptable salt thereof.

The compounds provided herein can modulate the activity of one or more sirtuins. For example, the compounds may modulate one or more of SIRT1, SIRT2, SIRT3, SIRT4, SIRT5, SIRT6, and SIRT7. In some embodiments, a compound provided herein may inhibit one or more sirtuins (e.g., two or more sirtuins). For example, a compound provided herein can inhibit SIRT1 and/or SIRT2. In some embodiments, the compound is

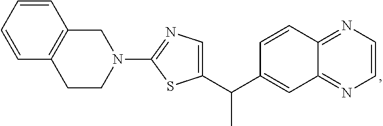 [SMILES: CC(c1ccc2nccnc2c1)c1cnc(N2CCc3ccccc3C2)s1]

or a pharmaceutically acceptable salt thereof. In some embodiments, a compound provided herein activates one or more sirtuins (e.g., two or more sirtuins). For example, a compound provided herein can activate SIRT1, SIRT2, and/or SIRT6. In some embodiments, the compound compound is:

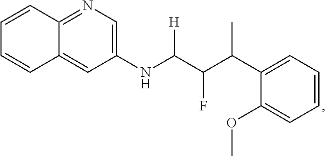 [SMILES: COc1ccccc1C(C)C(F)CNc1cnc2ccccc2c1]

or a pharmaceutically acceptable salt thereof.

In some embodiments, the methods above can further include administration of a therapeutically effective amount of a second compound provided herein.

In some embodiments, the methods above can further include administration of a therapeutically effective amount of an antiviral agent. Non-limiting examples of such agents include acyclovir, docosanol, ribarivin, interferons, and the like; cellulose acetate, carbopol and carrageenan, pleconaril, amantidine, rimantidine, fomivirsen, zidovudine, lamivudine, zanamivir, oseltamivir, brivudine, abacavir, adefovir, amprenavir, arbidol, atazanavir, atripla, cidofovir, combivir, edoxudine, efavirenz, emtricitabine, enfuvirtide, entecavir, famciclovir, fosamprenavir, foscarnet, fosfonet, ganciclovir, gardasil, ibacitabine, imunovir, idoxuridine, imiquimod, indinavir, inosine, integrase inhibitor, lamivudine, lopinavir, loviride, mk-0518, maraviroc, moroxydine, nelfinavir, nevirapine, nexavir, nucleotide and/or nucleoside analogues, oseltamivir, penciclovir, peramivir, podophyllotoxin, rimantadine, ritonavir, saquinavir, stavudine, tenofovir, tenofovir disoproxil, tipranavir, trifluridine, trizivir, tromantadine, truvada, valaciclovir, valganciclovir, vicriviroc, vidarabine, viramidine, zalcitabine, morpholino oligonucleotides, ribozyme, protease inhibitors, an assembly inhibitor, zidovudine, or a combination thereof.

Exemplary viruses that can be treated using the methods provided herein include: hepatitis B virus (HBV), woodchuck hepatitis virus, ground squirrel hepatitis virus, duck hepatitis B virus, heron hepatitis B virus, herpes simplex virus (HSV) types 1 and 2, varicella-zoster virus, cytomegalovirus (CMV), human cytomegalovirus (HCMV), Epstein-Barr virus (EBV), human herpesvirus 6 (variants A and B), human herpesvirus 7, human herpesvirus 8, Kaposi's sarcoma—associated herpes virus (KSHV), B virus, vaccinia virus, variola virus, smallpox virus, monkeypox virus, cowpox virus, camelpox virus, mousepox virus, raccoonpox viruses, molluscum contagiosum virus, orf virus, milker's nodes virus, bovin papullar stomatitis virus, sheeppox virus, goatpox virus, lumpy skin disease virus, fowlpox virus, canarypox virus, pigeonpox virus, sparrowpox virus, myxoma virus, hare fibroma virus, rabbit fibroma virus, squirrel fibroma viruses, swinepox virus, tanapox virus, Yabapox virus, dengue virus, hepatitis C virus (HCV), GB hepatitis viruses (GBV-A, GBV-B and GBV-C), West Nile virus, yellow fever virus, St. Louis encephalitis virus, Japanese encephalitis virus, Powassan virus, tick-borne encephalitis virus, Kyasanur Forest disease virus, Venezuelan equine encephalitis virus, chikungunya virus, Ross River virus, Mayaro virus, Sindbis virus, rubella virus, human immunodeficiency virus (HIV) types 1 and 2, human T cell leukemia virus (HTLV) types 1, 2, and 5, mouse mammary tumor virus (MMTV), Rous sarcoma virus (RSV), lentiviruses, severe acute respiratory syndrome (SARS) virus, Ebola virus, Marburg virus, rabies virus, vesicular stomatitis virus, Crimean-Congo hemorrhagic fever virus, Rift Valley fever virus, La Crosse virus, Hantaan virus, influenza virus (types A, B, and C), parainfluenza virus, respiratory syncytial virus (types A and B), measles virus, mumps virus, lymphocytic choriomeningitis virus, Junin virus, Machupo virus, Guanarito virus, Lassa virus, Ampari virus, Flexal virus, Ippy virus, Mobala virus, Mopeia virus, Latino virus, Parana virus, Pichinde virus, Tacaribe virus, Tamiami virus, canine parvovirus, parvovirus B19, porcine circovirus type 1 and 2, BFDV (Beak and Feather Disease Virus), chicken anaemia virus, simian virus 40 (SV40), JC virus, BK virus, Budgerigar fledgling disease virus, human papillomavirus, bovine papillomavirus (BPV) type 1, human adenovirus (HAdV-A, HAdV-B, HAdV-C, HAdV-D, HAdV-E, and HAdV-F), fowl adenovirus A, ovine adenovirus D, frog adenovirus, human orbivirus, human coltivirus, mammalian orthoreovirus, bluetongue virus, rotavirus A, rotaviruses (groups B to G), Colorado tick fever virus, aquareovirus A, cypovirus 1, Fiji disease virus, rice dwarf virus, rice ragged stunt virus, idnoreovirus 1, mycoreovirus 1, bursal disease virus, pancreatic necrosis virus, swine vesicular exanthema virus, rabbit hemorrhagic disease virus, Norwalk virus, Sapporo virus, human polioviruses (1-3), human coxsackieviruses A1-22, 24 (CA1-22 and CA24, CA23), human coxsackieviruses (B1-6 (CB1-6)), human echoviruses 1-7, 9, 11-27, 29-33, vilyuish virus, simian enteroviruses 1-18 (SEV1-18), porcine enteroviruses 1-11 (PEV1-11), bovine enteroviruses 1-2 (BEV1-2), hepatitis A virus, rhinoviruses, hepatoviruses, cardioviruses, aphthoviruses, and echoviruses.

In some embodiments, the virus is selected from the group consisting of: Human cytomegalovirus (HCMV), influenza virus, HIV, varicella zoster virus, ocular herpes simplex virus, adenovirus, hepatitis B, hepatitis C, papilloma virus, Human T-lymphotropic virus (HTLV), Epstein-Barr virus, adenovirus, poliovirus, human papillomavirus, Kaposi's sarcoma-associated herpes virus, herpes virus, and Merkel cell polyomavirus. For example, the virus can be selected from the group consisting of: HCMV, human papillomavirus, herpes virus, adenovirus, BK virus, JC virus, influenza virus, hepatitis B virus, hepatitis C virus, and HIV. In some embodiments, the virus is HCMV or influenza virus (e.g., influenza A).

In some embodiments, the virus is selected from the group consisting of Adenoviridae; Birnaviridae; Bunyaviridae; Caliciviridae; Circoviridae; Coronaviridae; Filoviridae; Hepadnaviridae; Herpesviridae; Flaviviridae; Orthomyxoviridae; Reoviridae; Retroviridae; Rhabdoviridae; Togaviridae; Paramyxoviridae; Parvoviridae; Pircornaviridae; Poxiviridae; Polyomaviridae; and Papillomaviridae.

A method of treating or preventing a viral infection in a subject is provided herein, the method including administering a therapeutically effective amount of a compound having the structure:

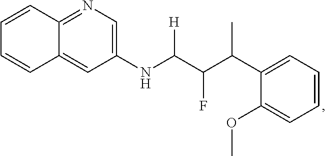 [SMILES: COc1ccccc1C(C)C(F)CNc1cnc2ccccc2c1]

or a pharmaceutically acceptable salt thereof. In some such embodiments, the virus is selected from the group consisting of HIV and hepatitis C.

Also provided herein is a method for treating cancer in a subject, the method comprising administering to the subject a therapeutically effective amount of a compound provided herein or a pharmaceutically acceptable salt thereof. Non-limiting examples of cancers include prostate cancer, bladder cancer, ovarian cancer, breast cancer, head and neck cancer, chronic lymphocytic leukemia, thymus cancer, hepatocellular carcinoma, colorectal cancer, colon cancer, skin cancer, pancreatic cancer, leukemia, lung cancer, glioblastoma, cervical cancer, lymphoma, and multiple myeloma.

The compounds provided herein can modulate the activity of one or more sirtuins. For example, the compounds may modulate one or more of SIRT1, SIRT2, SIRT3, SIRT4, SIRT5, SIRT6, and SIRT7. In some embodiments, a compound provided herein may inhibit one or more sirtuins (e.g., two or more sirtuins). In some such embodiments, the cancer can be selected from prostate cancer, bladder cancer, ovarian cancer, breast cancer, head and neck cancer, chronic lymphocytic leukemia, thymus cancer, hepatocellular carcinoma, colorectal cancer, and multiple myeloma. In some embodiments, a compound provided herein activates one or more sirtuins (e.g., two or more sirtuins). In some such embodiments, the cancer can be selected from colon cancer, skin cancer, breast cancer, prostate cancer, leukemia, pancreatic cancer, lung cancer, glioblastoma, cervical cancer and lymphoma.

In some embodiments, the methods of treating or preventing a cancer can further include administration of an additional chemotherapeutic agent, radiation, or a combination of two or more thereof.

Also provided herein is a method for treating a neurodegenerative disorder in a subject, the method comprising administering to the subject a therapeutically effective amount of a compound provided herein or a pharmaceutically acceptable salt thereof. Non-limiting examples of a neurodegenerative disorder include: Alzheimer's disease, Parkinson's disease, Huntington disease, amyotrophic lateral sclerosis, diffuse Lewy body disease, chorea-acanthocytosis, primary lateral sclerosis, multiple sclerosis, frontotemporal dementia, and Friedreich's ataxia.

The compounds provided herein can modulate the activity of one or more sirtuins. For example, the compounds may modulate one or more of SIRT1, SIRT2, SIRT3, SIRT4, SIRT5, SIRT6, and SIRT7. In some embodiments, a compound provided herein may inhibit one or more sirtuins (e.g., two or more sirtuins). In some such embodiments, the neurodegenerative disorder can be selected from Parkinson's disease, Huntington's disease, frontotemporal dementia, and multiple sclerosis. In some embodiments, a compound provided herein activates one or more sirtuins (e.g., two or more sirtuins). In some such embodiments, the neurodegenerative disorder can be selected from Alzheimer's disease, Parkinson's disease, multiple sclerosis, and amyotrophic lateral sclerosis.

Further provided herein is a method for treating or preventing inflammation in a subject, the method comprising administering to the subject a therapeutically effective amount of a compound provided herein or a pharmaceutically acceptable salt thereof. Non-limiting examples of an inflammation disorder include psoriasis, chronic obstructive pulmonary disorder, inflammatory uveitis, and atherosclerosis.

A method for treating or preventing a metabolic disease in a subject is also provided herein. The method includes administering to the subject a therapeutically effective amount of a compound provided herein or a pharmaceutically acceptable salt thereof. In some embodiments, the metabolic disease is selected from the group consisting of: diabetes, insulin resistance, metabolic syndrome, stenosis, cardiac ischemia, obesity, and lung thrombosis.

The compounds provided herein can also be used to promote the survival of a eukaryotic cell (e.g., a mammalian cell), the method comprising contacting the cell with a therapeutically effective amount of a compound provided herein or a pharmaceutically acceptable salt thereof. In some embodiments, the compound increases the lifespan of the cell. For example, the compound increases the cell's ability to resist stressors such as heat shock, osmotic stress, DNA damage, inadequate salt level, inadequate nitrogen level, or inadequate nutrient level. In some embodiments, the compound mimics the effect of nutrient restriction on the cell.

Also provided herein is a method for treating or preventing a disease or disorder associated with cell death or aging in a subject, the method comprising administering to the subject a therapeutically effective amount of a compound provided herein or a pharmaceutically acceptable salt thereof. Non-limiting examples of such disease or disorders include stroke, a cardiovascular disease, arthritis, high blood pressure, or Alzheimer's disease.

Further provided herein is a method for treating or preventing a blood coagulation disorder in a subject, the method comprising administering to the subject a therapeutically effective amount of a compound provided herein or a pharmaceutically acceptable salt thereof. Non-limiting examples of blood coagulation disorders include thromboembolism, deep vein thrombosis, pulmonary embolism, stroke, myocardial infarction, miscarriage, thrombophilia associated with anti-thrombin III deficiency, protein C deficiency, protein S deficiency, resistance to activated protein C, dysfibrinogenemia, fibrinolytic disorders, homo cystinuria, inflammatory disorders, myeloproliferative disorders, arteriosclerosis, angina, disseminated intravascular coagulation, thrombotic thrombocytopenic purpura, cancer metastasis, sickle cell disease, glomerular nephritis, drug induced thrombocytopenia, and re-occlusion during or after therapeutic clot lysis or procedures such as angioplasty or surgery.

A method for treating or preventing an ocular disease or disorder in a subject is provided herein. The method comprising administering to the subject a therapeutically effective amount of a compound provided herein or a pharmaceutically acceptable salt thereof. Examples of ocular diseases or disorders include vision impairment, glaucoma, optic neuritis, macular degeneration, and anterior ischemic optic neuropathy.

The compounds provided herein, or a pharmaceutically acceptable salt thereof, can be useful for treating or preventing a polyglutamine disease in a subject. The method can include administering to the subject a therapeutically effective amount of a compound provided herein. Non-limiting examples of a polyglutamine disease include spinobulbar muscular atrophy, Huntington's disease, dentatorubralpallidoluysian atrophy, spinocerebellar ataxia type 1, spinocerebellar ataxia type 2, spinocerebellar ataxia type 3, spinocerebellar ataxia type 6, spinocerebellar ataxia type 7, and spinocerebellar ataxia type 17. In some embodiments, the method further comprises administering a therapeutically effective amount of an HDAC I/II inhibitor.

Further provided herein is a method of modulating one or more sirtuins in a cell, the method comprising contacting a cell with an effective amount of a compound provided herein or a pharmaceutically acceptable salt thereof. In some embodiments, a compound provided herein inhibits two or more sirtuins. For example, the compound can inhibit two or more sirtuins selected from the group consisting of: SIRT1, SIRT2, SIRT3, SIRT4, SIRT5, SIRT6, and SIRT7. In some embodiments, the compound inhibits SIRT1 and/or SIRT2. For example, the compound can be:

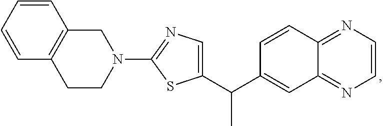 [SMILES: CC(c1ccc2nccnc2c1)c1cnc(N2CCc3ccccc3C2)s1]

or a pharmaceutically acceptable salt thereof. In some embodiments, a compound provided herein activates two or more sirtuins. For example, the compound can activate two or more sirtuins selected from the group consisting of: SIRT1, SIRT2, SIRT3, SIRT4, SIRT5, SIRT6, and SIRT7. In some embodiments, the compound activates two or more sirtuins selected from the group consisting of: SIRT1, SIRT2, and SIRT6. For example, the compound can be:

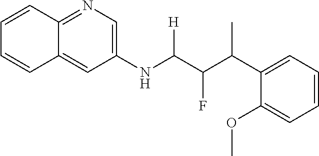 [SMILES: COc1ccccc1C(C)C(F)CNc1cnc2ccccc2c1]

or a pharmaceutically acceptable salt thereof.

In some embodiments, the contacting is in vitro. In some embodiments, the contacting is in vivo. For example, the contacting can include administering the compound to a subject such as a mammal (e.g., a human). The cell can be a virus-infected cell, a tumor cell, or a tumor cell infected with a virus (e.g., a tumor cell infected with HCMV).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 shows the antiviral effect of two sirtuin modulators on different viruses. MRC5 fibroblasts were infected with HCMV AD169 or influenza A WSN. Medium of the cells were replaced with fresh medium containing a sirtuin 1, 2, 6 activator (FIG. 1A), a sirtuin 1, 2 inhibitor (FIG. 1B) or vehicle (DMSO) at indicated concentrations at 2 hours after HCMV and 1 hour after influenza A infection. Virus yield was assayed at 96 hours after HCMV and 24 hours after influenza A infection.

### DETAILED DESCRIPTION

Provided herein are compounds useful for modulating one or more sirtuin enzymes. For example, the compounds provided herein can be used as activators or inhibitors of one or more sirtuin enzymes (i.e. SIRT1, SIRT2, SIRT3, SIRT4, SIRT5, SIRT6, and/or SIRT7). Sirtuin activity has been associated with a number of disease states, such as, viral infection, aging and stress, blood clotting disorders, cancer, cardiovascular diseases, diabetes, inflammation, neurodegenerative diseases, and obesity.

Sirtuins have been identified as innate viral restriction factors and it has been previously shown that drugs that modulate the activity of these enzymes block the production of multiple viruses in culture (see, e.g., WO 2012/106509). Sirtuins are NAD-dependent enzymes that regulate the activity of target proteins by post-translational modifications and thereby control key cellular events (see, e.g., Haigis and Sinclair (2010) Ann Rev Pathol Mech Dis 5:253-95). SIRT1, SIRT2, SIRT3 and SIRT6 have shown a detectable deacetylase activity in well-established biochemical assays that utilize recombinant SIRT1, SIRT2, SIRT3, and SIRT6 enzymes and fluorophore-conjugated acetyl-peptides. As described in the Examples below, the deacetylase activity of these sirtuins were used as a surrogate to identify compounds having broad-range antiviral activity.

Provided herein are methods for treating or preventing a viral infection in a subject. In some embodiments, the methods include administering a therapeutically effective amount of one or more of the compounds provided herein. In some embodiments, the compounds provided herein can inhibit virus production in a cell infected with a virus. In such embodiments, the cell is contacted with a therapeutically effective amount of one or more compounds provided herein.

A viral infection can include, for example, infection with a virus selected from Tables 1 and 2, which include: Human cytomegalovirus (HCMV) (a genus member of Herpesviridae family), influenza virus (e.g., Influenza A), HIV (e.g., HIV-1), adenovirus, varicella zoster viruses, herpes simplex virus (types 1 and 2), adenovirus, hepatitis B (“hepatocellular carcinoma cells expressing hepatitis B virus antigens”), hepatitis C, papilloma viruses, Human T-lymphotropic virus (HTLV) (e.g., HTLV-1), Epstein-Barr virus, poliovirus, human papillomavirus, Kaposi's sarcoma-associated herpes virus, herpes virus, Merkel cell polyomavirus. In some embodiments, the virus can be a virus implicated in the causation or progression of cancer, for example, human papillomavirus, Kaposi's sarcoma-associated herpes virus, herpes virus (Herpesviridae family), Epstein-Barr virus (a member of Herpesviridae), Merkel cell polyomavirus, hepatitis B virus, hepatitis C virus, and HTLV.

TABLE 1
Families of Enveloped Viruses

| Virus Family | Members |
|---|---|
| Hepadnavirus | hepatitis B virus (HBV), woodchuck hepatitis virus, ground |
| (Hepadnaviridae) | squirrel hepatitis virus, duck hepatitis B virus, heron hepatitis B |
| | virus |
| Herpesvirus | herpes simplex virus (HSV) types 1 and 2, varicella-zoster virus, |
| (Herpesviridae) | cytomegalovirus (CMV), human cytomegalovirus (HCMV), |
| | Epstein-Barr virus (EBV), human herpesvirus 6 (variants A and B), |
| | human herpesvirus 7, human herpesvirus 8, Kaposi's sarcoma - |
| | associated herpes virus (KSHV), B virus |
| Poxvirus | vaccinia virus, variola virus, smallpox virus, monkeypox virus, |
| (Poxviridae) | cowpox virus, camelpox virus, mousepox virus, raccoonpox |
| | viruses, molluscum contagiosum virus, orf virus, milker's nodes |
| | virus, bovin papullar stomatitis virus, sheeppox virus, goatpox |
| | virus, lumpy skin disease virus, fowlpox virus, canarypox virus, |
| | pigeonpox virus, sparrowpox virus, myxoma virus, hare fibroma |
| | virus, rabbit fibroma virus, squirrel fibroma viruses, swinepox |
| | virus, tanapox virus, Yabapox virus |
| Flavivirus | dengue virus, hepatitis C virus (HCV), GB hepatitis viruses (GBV- |
| (Flaviviridae) | A, GBV-B and GBV-C), West Nile virus, yellow fever virus, |
| | St. Louis encephalitis virus, Japanese encephalitis virus, Powassan |
| | virus, tick-borne encephalitis virus, Kyasanur Forest disease virus |
| Togavirus | Venezuelan equine encephalitis virus, chikungunya virus, Ross |
| (Togaviridae) | River virus, Mayaro virus, Sindbis virus, rubella virus |
| Retrovirus | human immunodeficiency virus (HIV) types 1 and 2, human T cell |
| (Retroviridae) | leukemia virus (HTLV) types 1, 2, and 5, mouse mammary tumor |
| | virus (MMTV), Rous sarcoma virus (RSV), lentiviruses |
| Coronavirus | severe acute respiratory syndrome (SARS) virus |
| (Coronaviridae) | |
| Filovirus | Ebola virus, Marburg virus |
| (Filoviridae) | |
| Rhabdovirus | rabies virus, vesicular stomatitis virus |
| (Rhabdoviridae) | |
| Bunyavirus | Crimean-Congo hemorrhagic fever virus, Rift Valley fever virus, |
| (Bunyaviridae) | La Crosse virus, Hantaan virus |
| Orthomyxovirus | influenza virus (types A, B, and C) |
| (Orthomyxoviridae) | |
| Paramyxovirus | parainfluenza virus, respiratory syncytial virus (types A and B), |
| (Paramyxoviridae) | measles virus, mumps virus |
| Arenavirus | lymphocytic choriomeningitis virus, Junin virus, Machupo virus, |
| (Arenaviridae) | Guanarito virus, Lassa virus, Ampari virus, Flexal virus, Ippy |
| | virus, Mobala virus, Mopeia virus, Latino virus, Parana virus, |
| | Pichinde virus, Tacaribe virus, Tamiami virus |

TABLE 2
Families of Non-Enveloped (Naked) Viruses

| Virus Family | Members |
|---|---|
| Parvovirus | canine parvovirus, parvovirus B19 |
| (Parvoviridae) | |
| Circovirus | porcine circovirus type 1 and 2, BFDV (Beak and Feather Disease |
| (Circoviridae) | Virus), chicken anaemia virus |
| Polyomavirus | simian virus 40 (SV40), JC virus, BK virus, Budgerigar fledgling |
| (Polyomaviridae) | disease virus |
| Papillomavirus | human papillomavirus, bovine papillomavirus (BPV) type 1 |
| (Papillomaviridae) | |
| Adenovirus | human adenovirus (HAdV-A, HAdV-B, HAdV-C, HAdV-D, |
| (Adenoviridae) | HAdV-E, and HAdV-F), fowl adenovirus A, ovine adenovirus D, |
| | frog adenovirus |
| Reovirus | human orbivirus, human coltivirus, mammalian orthoreovirus, |
| (Reoviridae) | bluetongue virus, rotavirus A, rotaviruses (groups B to G), |
| | Colorado tick fever virus, aquareovirus A, cypovirus 1, Fiji |
| | disease virus, rice dwarf virus, rice ragged stunt virus, |
| | idnoreovirus 1, mycoreovirus 1 |
| Birnavirus | bursal disease virus, pancreatic necrosis virus |
| (Birnaviridae) | |
| Calicivirus | swine vesicular exanthema virus, rabbit hemorrhagic disease |
| (Caliciviridae) | virus, Norwalk virus, Sapporo virus |
| Picornavirus | human polioviruses (1-3), human coxsackieviruses A1-22, 24 |
| (Picornaviridae) | (CA1-22 and CA24, CA23 = echovirus 9), human |
| | coxsackieviruses (B1-6 (CB1-6)), human echoviruses 1-7, 9, 11- |
| | 27, 29-33, vilyuish virus, simian enteroviruses 1-18 (SEV1-18), |
| | porcine enteroviruses 1-11 (PEV1-11), bovine enteroviruses 1-2 |
| | (BEV1-2), hepatitis A virus, rhinoviruses, hepatoviruses, |
| | cardioviruses, aphthoviruses, echoviruses |

In some embodiments, a viral infection can be treated or prevented by activation of one or more sirtuins (e.g., SIRT1 activation). Accordingly, compounds provided herein may be useful in the treatment and/or prevention of viruses such as HIV and hepatitis C, which have been linked with attenuation of sirtuin activity.

Viral infections can also include the following non-limiting examples: Adenoviruses (Adenoviridae family) of which there are 57 known human adenovirus types (HAdV-1 to 57) in seven species (human adenovirus A to G) (e.g. HAdV-B type 7 (respiratory disease) or HAdV-F type 40 (gastroenteritis)); severe acute respiratory syndrome (SARS) virus (Coronaviridae); simian virus 40 (SV40), JC virus, BK virus, Budgerigar fledgling disease virus (Polyomaviridae); bursal disease virus, pancreatic necrosis virus (Birnaviridae); Poxviruses (Poxviridae family) including cowpox, monkeypox, smallpox (variola), and vaccinia; Parvoviruses (Parvoviridae family) including parvovirus B19 (fifth disease); Paramyxoviruses (Paramyxoviridae family) including measles, mumps, subfamily Pneumovirinae (e.g., human respiratory syncytial virus); Reoviruses (Reoviridae family) including rotavirus; Picornaviruses (Picornaviridae family) including human rhinoviruses, poliomyelitis, enteroviruses, and hepatitis A virus; Togaviruses (Togaviridae family) including rubella virus and Ross River virus; Orthomyxoviruses (Orthomyxoviridae family) including influenza viruses A, B, and C, avian influenza, and swine influenza; Caliciviruses (Caliciviridae family) including noroviruses and sapoviruses; Rhabdoviruses (Rhabdoviridae family) including rabies virus; Flaviviruses (Flaviviridae family) including hepatitis C, St. Louis encephalitis, West Nile encephalitis, Japanese encephalitis; Retroviruses including HIV, Hepatitis B virus, and Human T-lymphotropic virus (HTLV); Viruses that cause viral hemorrhagic fever such as five families of RNA viruses, including Arenaviridae (e.g., Lassa fever and Argentine hemorrhagic fever), Bunyaviridae (e.g., Hantavirus genus and Rift Valley fever), Filoviridae (e.g., Ebola and Marburg viruses), Flaviviridae (e.g., dengue fever, yellow fever and Omsk hemorrhagic fever), and Rhabdoviridae.

In some embodiments, the virus can be a virus which infects non-human subjects. For example, the virus may infect livestock such as cows, pigs, horses, sheep, goats, chickens, and geese, or the virus may infect domestic animals such as dogs and cats. Non-limiting examples of such viruses include Circoviruses (e.g., porcine circovirus types 1 and 2 and goose circovirus); Arteriviruses (e.g., equine arteritis virus (EAV), porcine reproductive and respiratory syndrome virus (PRRSV), simian hemorrhagic fever virus (SHFV)); African swine fever virus; swine influenza; avian influenza; Poxviridae (e.g., myxomatosis (pox virus infection of rabbits), pseudocowpox, buffalopox, Orf virus, bovine papular stomatitis virus, and Yaba monkey tumor virus); foot-and-mouth disease virus; Pestiviruses (e.g., viruses that cause Classical swine fever (CSF) and bovine viral diarrhea (BVD)); Coronaviruses; Bluetongue virus (BTV); Paramyxoviruses (e.g., rinderpest virus (cattle) and canine distemper); Pseudorabies (including infections caused by Suid herpesvirus 1 (SuHV-1) a.k.a. pseudorabies virus (PRV)); fog fever (i.e. acute bovine pulmonary edema and emphysema (ABPEE)); viruses that cause calf pneumonia (parainfluenza III, bovine respiratory syncytial virus, infectious bovine rhinotracheitis (IBR)); epizootic hemorrhagic disease (EHD); Akabane virus; and Schmallenberg virus. In some embodiments, the virus can affect cats. For example, the virus can include feline viral rhinotracheitis (feline herpes virus); feline immunodeficiency virus (a retrovirus); feline enteric coronavirus (FECV); feline calicivirus; feline coronavirus; feline leukemia virus (FeLV) (a retrovirus); and feline panleukopenia (FPV) (a.k.a. feline distemper). In some embodiments, the virus can affect dogs. For example, the virus can include canine influenza; canine coronavirus; canine herpes virus; canine distemper virus; canine parvovirus; infectious canine hepatitis (including infections caused by canine adenovirus type-1 (CAV-1)); canine minute virus; and parainfluenza.

A method for treating or preventing a viral infection in a subject provided herein can include administering a therapeutically effective amount of a sirtuin activator such as a compound having the structure:

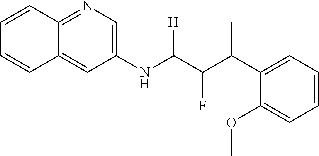 [SMILES: COc1ccccc1C(C)C(F)CNc1cnc2ccccc2c1]

or a pharmaceutically acceptable salt form thereof. In some embodiments, the viral infection is caused by a virus selected from HIV and hepatitis C.

An antiviral agent can also be administered in conjunction with the compounds and the methods described herein. The agent can be any therapeutic agent useful in the treatment of a viral infection. For example, an antiviral agent can include acyclovir, docosanol, ribarivin, interferons, and the like; cellulose acetate, carbopol and carrageenan, pleconaril, amantidine, rimantidine, fomivirsen, zidovudine, lamivudine, zanamivir, oseltamivir, brivudine, abacavir, adefovir, amprenavir, arbidol, atazanavir, atripla, cidofovir, combivir, edoxudine, efavirenz, emtricitabine, enfuvirtide, entecavir, famciclovir, fosamprenavir, foscarnet, fosfonet, ganciclovir, gardasil, ibacitabine, imunovir, idoxuridine, imiquimod, indinavir, inosine, integrase inhibitor, lamivudine, lopinavir, loviride, mk-0518, maraviroc, moroxydine, nelfinavir, nevirapine, nexavir, nucleotide and/or nucleoside analogues, oseltamivir, penciclovir, peramivir, podophyllotoxin, rimantadine, ritonavir, saquinavir, stavudine, tenofovir, tenofovir disoproxil, tipranavir, trifluridine, trizivir, tromantadine, truvada, valaciclovir, valganciclovir, vicriviroc, vidarabine, viramidine, zalcitabine, morpholino oligonucleotides, ribozyme, protease inhibitors, an assembly inhibitor (e.g., rifampicin), zidovudine, or a combination or two or more antiviral agents.

In some embodiments, a compound provided herein can be administered before, after, or simultaneously with the administration or one or more antiviral agents.

Compounds provided herein may also be used in the treatment of cancer in a subject. Such methods can include the administration of a therapeutically effective amount of one or more compounds provided herein to the subject. Modulation of sirtuin enzymes has been associated with the treatment of a variety of cancers, including prostate cancer, bladder cancer, ovarian cancer, breast cancer (e.g., breast cancer with mutated BRCA1), head and neck cancer, chronic lymphocytic leukemia, thymus cancer, hepatocellular carcinoma, colorectal cancer, colon cancer, skin cancer, pancreatic cancer, leukemia, lung cancer, glioblastoma, cervical cancer, lymphoma, and multiple myeloma. In some embodiments, a sirtuin inhibitor such as a SIRT1 or SIRT2 inhibitor can be administered to a subject. For example, a sirtuin inhibitor can be used to treat cancers selected from prostate cancer, bladder cancer, ovarian cancer, breast cancer (with mutated BRCA1), head and neck cancer, chronic lymphocytic leukemia, thymus cancer, hepatocellular carcinoma, colorectal cancer, and multiple myeloma. In some embodiments, a sirtuin activator such as a SIRT1 or SIRT2 activator can be administered to a subject. For example, a sirtuin activator can be used to treat cancers selected from colon cancer, skin cancer, breast cancer, prostate cancer, leukemia, pancreatic cancer, lung cancer, glioblastoma, cervical cancer, and lymphoma (e.g., Burkitt lymphoma).

The treatment of cancer, as provided herein, can be combined with existing methods of treating cancers, for example by chemotherapy, radiation, or surgery (e.g., oophorectomy). In some embodiments, compounds provided herein can be administered in conjunction with another anticancer agent. For example, a compound provided herein may be administered with paclitaxel, etopisode, gemcitabine, an alkylating agent (e.g., Cytoxan and leukeran), anthracyclines (e.g., Daunomycin and Adriamycin), topoisomerase inhibitors (e.g., doxorubicin). Other anticancer agents include docetaxel, daunorubicin, epirubicin, fluorouracil, melphalan, cisplatin, carboplatin, cyclophosphamide, mitomycin, methotrexate, mitoxantrone, vinblastine, vincristine, ifosfamide, teniposide, etoposide, bleomycin, leucovorin, taxol, herceptin, avastin, cytarabine, dactinomycin, interferon alpha, streptozocin, prednisolone, irinotecan, sulindac, 5-fluorouracil, capecitabine and procarbazine. The compounds provided herein can be administered before, during, or after another anticancer agent or treatment.

Sirtuin activity has been implicated in many neurodegenerative disorders. For example, sirtuin activation or upregulation has been found to have a neuroprotective effect in patients diagnosed with Alzheimer's disease, Parkinson's disease, multiple sclerosis, and amyotrophic lateral sclerosis (see, e.g., Araki, T. et al. Science (2004) 305:1010-1013; and Kim, D. et al. EMBO J. (2007) 26(13):3169-3179). Accordingly, the compounds provided herein can be used to treat a neurodegenerative disorder in a subject. Neurodegenerative disorders can include Alzheimer's disease (AD), Parkinson's disease (PD), Huntington disease (HD), amyotrophic lateral sclerosis (ALS; Lou Gehrig's disease), diffuse Lewy body disease, chorea-acanthocytosis, primary lateral sclerosis, multiple sclerosis (MS), frontotemporal dementia, and Friedreich's ataxia. In some embodiments, a sirtuin inhibitor such as a SIRT1 inhibitor can be administered to a subject. For example, a sirtuin inhibitor can be used to treat a neurodegenerative disorder selected from multiple sclerosis, Parkinson's disease, Huntington's disease, and frontotemporal dementia.

The compounds provided herein can be used in methods of treating inflammation in a subject. For example, sirtuin activators can be used to treat or prevent a disease or disorder associated with inflammation. When used prophylactically, the compounds provided herein can be administered in advance of any inflammatory response or symptom. Administration of the compounds may prevent or attenuate inflammatory responses or symptoms. For example, activation of SIRT1 has been found to modulate the nFκB pathway. In some embodiments, a SIRT1 activator is administered to the patient.

Exemplary inflammatory conditions include, for example, multiple sclerosis, rheumatoid arthritis, psoriatic arthritis, degenerative joint disease, spondouloarthropathies, gouty arthritis, systemic lupus erythematosus, juvenile arthritis, rheumatoid arthritis, osteoarthritis, osteoporosis, diabetes (e.g., insulin dependent diabetes mellitus or juvenile onset diabetes), menstrual cramps, cystic fibrosis, inflammatory bowel disease, irritable bowel syndrome, Crohn's disease, mucous colitis, ulcerative colitis, gastritis, esophagitis, pancreatitis, peritonitis, shock, ankylosing spondylitis, gastritis, conjunctivitis, pancreatis (acute or chronic), multiple organ injury syndrome (e.g., secondary to septicemia or trauma), myocardial infarction, atherosclerosis, stroke, reperfusion injury (e.g., due to cardiopulmonary bypass or kidney dialysis), acute glomerulonephritis, vasculitis, thermal injury (i.e., sunburn), necrotizing enterocolitis, granulocyte transfusion associated syndrome, and/or Sjogren's syndrome. Exemplary inflammatory conditions of the skin include, for example, eczema, atopic dermatitis, contact dermatitis, urticaria, scleroderma, psoriasis, and dermatosis with acute inflammatory components. In some embodiments, the inflammatory condition is selected from psoriasis, chronic obstructive pulmonary disorder (COPD), inflammatory uveitis, and atherosclerosis.

In some embodiments, a compound provided herein may be administered in combination with other compounds useful for treating or preventing inflammation. Exemplary anti-inflammatory agents include, for example, steroids (e.g., Cortisol, cortisone, fludrocortisone, prednisone, 6α-methylprednisone, triamcinolone, betamethasone or dexamethasone); nonsteroidal anti-inflammatory drugs (NSAIDS (e.g., aspirin, acetaminophen, tolmetin, ibuprofen, mefenamic acid, piroxicam, nabumetone, rofecoxib, celecoxib, etodolac or nimesulide). In some embodiments, the other therapeutic agent is an antibiotic (e.g., vancomycin, penicillin, amoxicillin, ampicillin, cefotaxime, ceftriaxone, cefixime, rifampinmetronidazole, doxycycline or streptomycin).

The compounds provided herein can also be used to treat and/or prevent metabolic diseases. For example, activation of SIRT1 and SIRT6 has been shown to be useful in the treatment of diabetes. This is due in part because SIRT1 is a positive regulator of insulin secretion while SIRT6 activation has been correlated with enhanced glucose intolerance. Non-limiting examples of metabolic diseases include diabetes, insulin resistance, metabolic syndrome, stenosis, cardiac ischemia, obesity, and lung thrombosis. In some embodiments, the metabolic disease is diabetes. In some embodiments the compound is an activator of SIRT1, SIRT2 and/or SIRT6.

Also provided herein are methods relating to the treatment of disorders related to aging and/or stress. For example, the compounds provided herein can be used to promote the survival of a eukaryotic cell (e.g., a mammalian cell) by contacting the cell with a therapeutically effective amount of one or more compounds provided herein. For example, the compounds can be used to increase the lifespan of the cell or increase the cell's ability to resist stress such as heat shock, DNA damage, inadequate salt levels, inadequate nitrogen levels, and/or inadequate nutrient levels. In some embodiments, the compounds provided herein can mimic the effect of nutrient restriction on the cell.

In some embodiments, cells that are intended to be preserved for long periods of time may be treated with a sirtuin activator. The cells may be in suspension (e.g., blood cells, serum, biological growth media, etc.) or in tissues or organs. For example, blood collected from an individual for purposes of transfusion may be treated with a sirtuin activator to preserve the blood cells for longer periods of time. Additionally, blood to be used for forensic purposes may also be preserved using a sirtuin activator. Other cells that may be treated to extend their lifespan or protect against apoptosis include cells for consumption, e.g., cells from non-human mammals (such as meat) or plant cells (such as vegetables). Sirtuin activators may also be applied during developmental and growth phases in mammals, plants, insects or microorganisms, in order to, for example, alter, retard or accelerate the developmental and/or growth process.

In some embodiments, sirtuin activators may be used to treat cells useful for transplantation or cell therapy, including, for example, solid tissue grafts, organ transplants, cell suspensions, stem cells, bone marrow cells, etc. The cells or tissue may be an autograft, an allograft, a syngraft or a xenograft. The cells or tissue may be treated with the sirtuin activator prior to administration/implantation, concurrently with administration/implantation, and/or post administration/implantation into a subject. The cells or tissue may be treated prior to removal of the cells from the donor individual, ex vivo after removal of the cells or tissue from the donor individual, or post implantation into the recipient.

In some embodiments, cells may be treated with a sirtuin activator by administering a compound provided herein to a subject, for example, to increase the lifespan or prevent apoptosis of the cells. For example, skin can be protected from aging (e.g., developing wrinkles, loss of elasticity, etc.) by treating skin or epithelial cells with a sirtuin activator. In some embodiments, compounds provided herein (e.g., sirtuin activators) may be used for the treatment of wounds and/or burns to promote healing, including, for example, first-, second- or third-degree burns and/or a thermal, chemical or electrical burns.

Compounds provided herein can be used to treat or prevent a blood coagulation disorder in a subject. Examples of such disorders include thromboembolism, deep vein thrombosis, pulmonary embolism, stroke, myocardial infarction, miscarriage, thrombophilia associated with anti-thrombin III deficiency, protein C deficiency, protein S deficiency, resistance to activated protein C, dysfibrinogenemia, fibrinolytic disorders, homo cystinuria, inflammatory disorders, myeloproliferative disorders, arteriosclerosis, angina, disseminated intravascular coagulation, thrombotic thrombocytopenic purpura, cancer metastasis, sickle cell disease, glomerular nephritis, drug induced thrombocytopenia, and re-occlusion during or after therapeutic clot lysis or procedures such as angioplasty or surgery.

Sirtuin activity has been associated with ocular diseases and disorders. Accordingly, provided herein is a method of treating or preventing an ocular disease or disorder in a subject including administering a therapeutically effective amount of a compound provided herein. Examples of ocular diseases and disorders include vision impairment, glaucoma, optic neuritis, macular degeneration, and anterior ischemic optic neuropathy. In some embodiments, the compound is a sirtuin activator (e.g., a SIRT1 activator).

In some embodiments, compounds provided herein can be used to treat or prevent a polyglutamine disease. Huntington's Disease (HD) and Spinocerebellar ataxia type 1 (SCA1) are just two examples of a class of genetic diseases caused by dynamic mutations involving the expansion of triplet sequence repeats. In reference to this common mechanism, these disorders are called trinucleotide repeat diseases. At least 14 such diseases are known to affect human beings. Nine of them, including SCA1 and Huntington's disease, have CAG as the repeated sequence. Since CAG codes for an amino acid called glutamine, these nine trinucleotide repeat disorders are collectively known as polyglutamine diseases.

Although the genes involved in different polyglutamine diseases have little in common, the disorders they cause follow a strikingly similar course. Each disease is characterized by a progressive degeneration of a distinct group of nerve cells. The major symptoms of these diseases are similar, although not identical, and usually affect people in midlife. Given the similarities in symptoms, the polyglutamine diseases are hypothesized to progress via common cellular mechanisms.

Non-limiting examples of polyglutamine diseases include spinobulbar muscular atrophy (Kennedy disease), Huntington's disease, dentatorubralpallidoluysian atrophy (Haw River syndrome), spinocerebellar ataxia type 1, spinocerebellar ataxia type 2, spinocerebellar ataxia type 3 (Machado-Joseph disease), spinocerebellar ataxia type 6, spinocerebellar ataxia type 7, and spinocerebellar ataxia type 17.

In some embodiments, compounds provided herein can be administered in conjunction with an HDAC I or HDAC II inhibitor. Examples of HDAC I/II inhibitors include hydroxamic acids, cyclic peptides, benzamides, short-chain fatty acids, and depudecin.

In any of the methods described herein, the method can further include the administration of a second sirtuin modulator. For example, the methods provided herein can include the administration of two or more compounds provided herein.

Also provided herein is a method of modulating one or more sirtuins in a cell. The method can include contacting the cell with a therapeutically effective amount of a compound provided herein. In some embodiments, the compound provided herein inhibits sirtuin activity. In some embodiments, the compound provided herein activates sirtuin activity. A sirtuin can include one or more of SIRT1, SIRT2, SIRT3, SIRT4, SIRT5, SIRT6, and SIRT7. The method of modulating one or more sirtuins in a cell may be performed by contacting the cell with a compound provided herein, in vitro, thereby inducing modulating of one or more sirtuins of a cell in vitro. Uses of such an in vitro methods of modulating one or more sirtuins include, but are not limited to use in a screening assay (for example, wherein a compound provided herein is used as a positive control or standard compared to compounds of unknown activity or potency in modulating one or more sirtuins). In some embodiments, the cell is a tumor cell or a virus-infected cell.

The method of modulating one or more sirtuins in a cell may be performed, for example, by contacting a cell with a compound provided herein, in vivo, thereby modulating one or more sirtuins in a subject in vivo. The contacting is achieved by causing a compound provided herein to be present in the subject in an amount effective to achieve modulation of one or more sirtuins. This may be achieved, for example, by administering an effective amount of a compound provided herein to a subject. Uses of such an in vivo method of modulating one or more sirtuins include, but are not limited to use in methods of treating a disease or condition, wherein modulating one or more sirtuins is beneficial. In some such embodiments, one or more sirtuins are modulated in a tumor cell (e.g., a tumor cell infected with HCMV), for example in a patient suffering from cancer. In some embodiments, one or more sirtuins are modulated in a virus-infected cell, for example, in a patient infected with the virus. Such methods can be performed by administering an effective amount of a compound provided herein to a subject in need of such treatment.

In some of the above embodiments, the modulating of one or more sirtuins includes inhibiting one or more sirtuins. In some embodiments, the compound may inhibit two or more sirtuins. For example, inhibiting SIRT1 and SIRT2. In some of the above embodiments, the modulating of one or more sirtuins includes activating one or more sirtuins. In some embodiments, the compound may activate two or more sirtuins. For example, activating SIRT1, SIRT2, and/or SIRT6.

### Compounds and Compositions

Provided herein are compounds useful for modulating one or more sirtuins. Such compounds include:

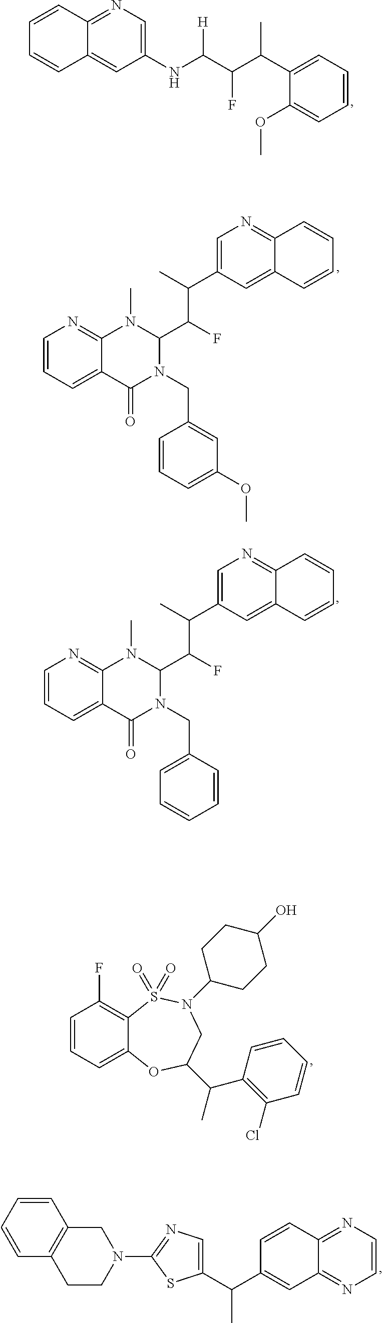

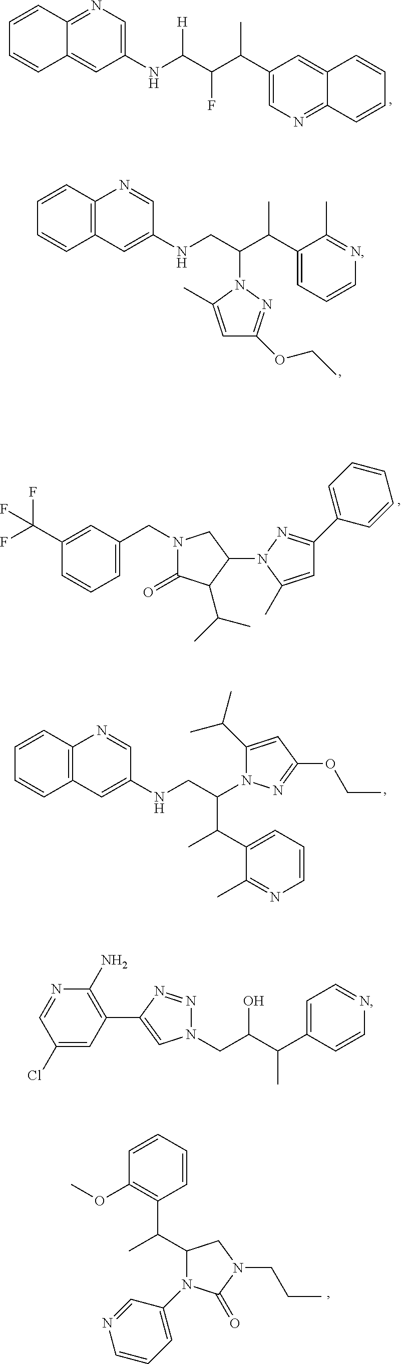

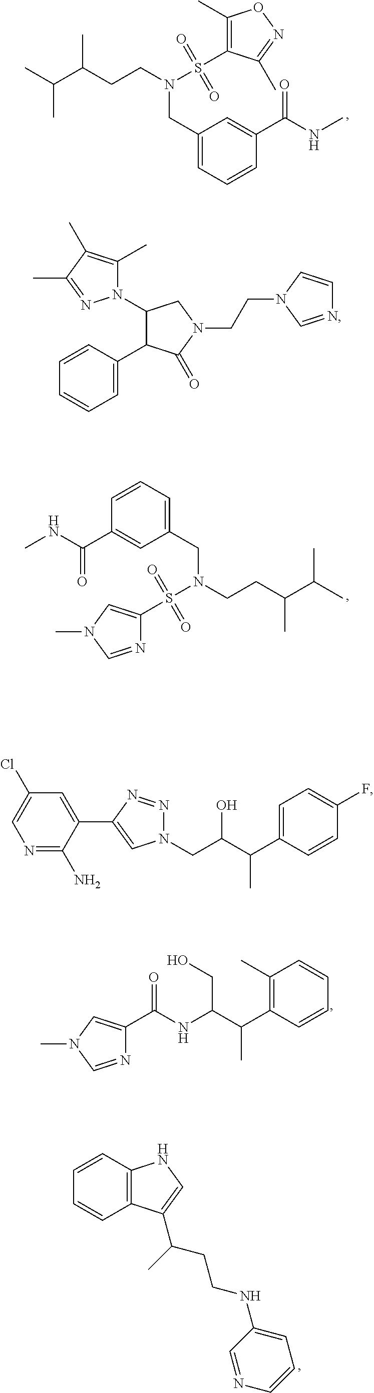

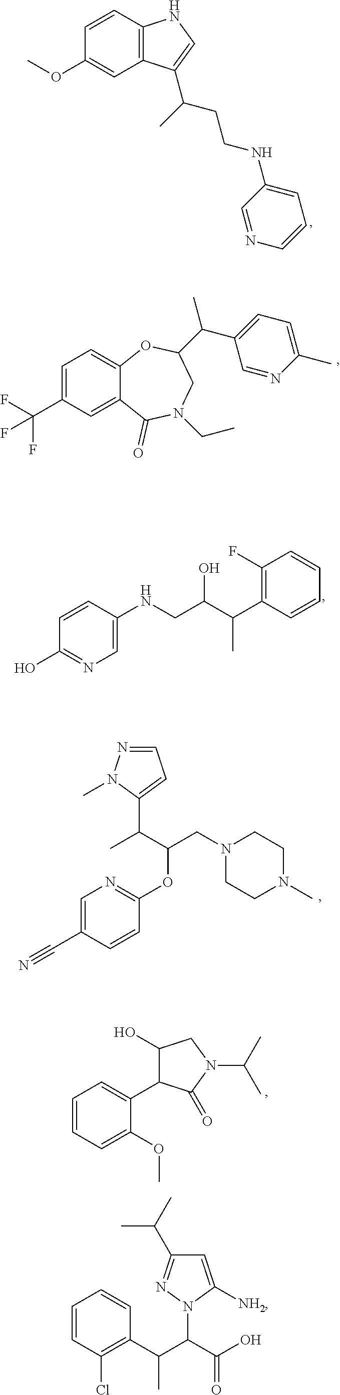

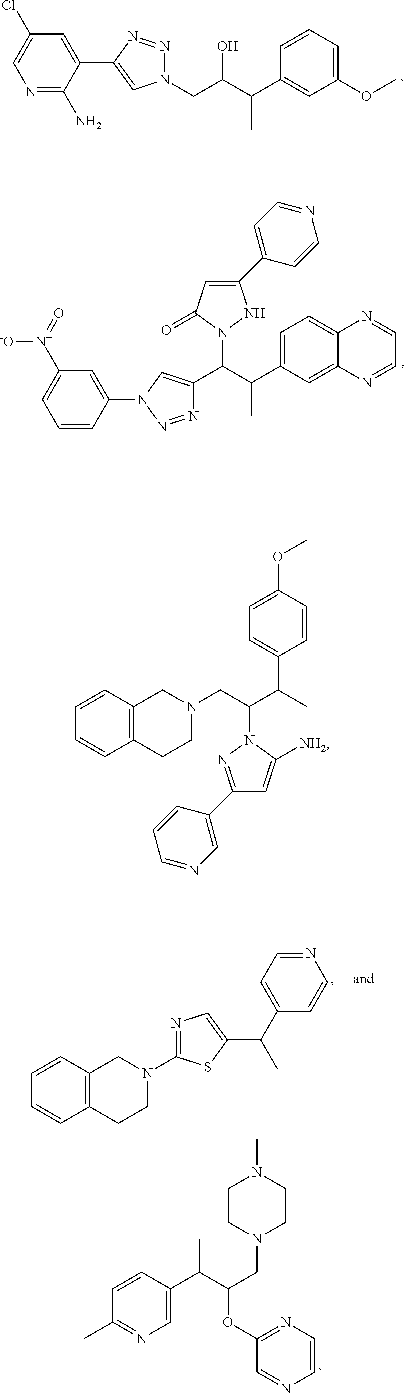

or a pharmaceutically acceptable salt form thereof.

In some of the above embodiments, the modulating of one or more sirtuins includes inhibiting one or more sirtuins. In some embodiments, the compound may inhibit two or more sirtuins. For example, inhibiting SIRT1 and/or SIRT2. In some embodiments, a compound provided herein is a sirtuin inhibitor such as

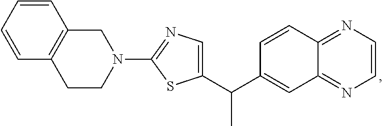 [SMILES: CC(c1ccc2nccnc2c1)c1cnc(N2CCc3ccccc3C2)s1]

or a pharmaceutically acceptable salt thereof. In some of the above embodiments, the modulating of one or more sirtuins includes activating one or more sirtuins. In some embodiments, the compound may activate two or more sirtuins. For example, activating SIRT1, SIRT2, and/or SIRT6. In some embodiments, a compound provided herein is a sirtuin activator such as

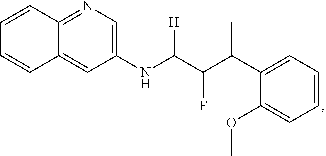 [SMILES: COc1ccccc1C(C)C(F)CNc1cnc2ccccc2c1]

or a pharmaceutically acceptable salt form thereof.

A compound provided herein, including a pharmaceutically acceptable salt thereof, can be purchased commercially or prepared using known organic synthesis techniques.

The methods provided herein include the manufacture and use of pharmaceutical compositions, which include compounds provided herein and one or more pharmaceutically acceptable carriers. Also provided herein are the compositions themselves.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

A pharmaceutical composition is typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol, or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates, or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Pharmaceutical compositions suitable for injection can include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. The composition should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent that delays absorption, for example, aluminum monostearate and gelatin.

Sterile injectable solutions can be prepared by incorporating a compound provided herein in the required amount in an appropriate solvent with one or a combination of ingredients enumerated above, as required, followed by filtered sterilization. Generally, dispersions are prepared by incorporating a compound provided herein into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of a compound provided herein plus any additional desired ingredient from a previously sterile-filtered solution thereof.

Oral compositions generally include an inert diluent or an edible carrier. For the purpose of oral therapeutic administration, a compound provided herein can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the compounds provided herein can be formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical composition may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular patient, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Dosage forms or compositions containing a compound as described herein in the range of 0.005% to 100% with the balance made up from non-toxic carrier may be prepared. Methods for preparation of these compositions are known to those skilled in the art. The contemplated compositions may contain 0.001%-100% of a compound provided herein, in one embodiment 0.1-95%, in another embodiment 75-85%.

The pharmaceutical compositions can be included in a container, pack, or dispenser together with instructions for administration.

As described above, the preparations of one or more compounds provided herein may be given orally, parenterally, topically, or rectally. They are, of course, given by forms suitable for each administration route. For example, they are administered in tablets or capsule form, by injection, inhalation, eye lotion, ointment, suppository, infusion; topically by lotion or ointment; and rectally by suppositories. In some embodiments, administration is oral.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrastemal injection, and infusion.

Actual dosage levels of the active ingredients in the pharmaceutical compositions provided herein may be varied so as to obtain an amount of the active ingredient which is effective to achieve the desired therapeutic response for a particular patient, composition, and mode of administration, without being toxic to the patient.

The concentration of a compound provided herein in a pharmaceutically acceptable mixture will vary depending on several factors, including the dosage of the compound to be administered, the pharmacokinetic characteristics of the compound(s) employed, and the route of administration. In some embodiments, the compositions provided herein can be provided in an aqueous solution containing about 0.1-10% w/v of a compound disclosed herein, among other substances, for parenteral administration. Typical dose ranges can include from about 0.01 to about 50 mg/kg of body weight per day, given in 1-4 divided doses. Each divided dose may contain the same or different compounds. The dosage will be a therapeutically effective amount depending on several factors including the overall health of a patient, and the formulation and route of administration of the selected compound(s).

Dosage forms or compositions containing a compound as described herein in the range of 0.005% to 100% with the balance made up from non-toxic carrier may be prepared. Methods for preparation of these compositions are known to those skilled in the art. The contemplated compositions may contain 0.001%-100% active ingredient, in one embodiment 0.1-95%, in another embodiment 75-85%. Although the dosage will vary depending on the symptoms, age and body weight of the patient, the nature and severity of the disorder to be treated or prevented, the route of administration and the form of the drug, in general, a daily dosage of from 0.01 to 2000 mg of the compound is recommended for an adult human patient, and this may be administered in a single dose or in divided doses. The amount of active ingredient which can be combined with a carrier material to produce a single dosage form will generally be that amount of the compound which produces a therapeutic effect.

The pharmaceutical composition may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular patient, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

The precise time of administration and/or amount of the composition that will yield the most effective results in terms of efficacy of treatment in a given patient will depend upon the activity, pharmacokinetics, and bioavailability of a particular compound, physiological condition of the patient (including age, sex, disease type and stage, general physical condition, responsiveness to a given dosage, and type of medication), route of administration, etc. However, the above guidelines can be used as the basis for fine-tuning the treatment, e.g., determining the optimum time and/or amount of administration, which will require no more than routine experimentation consisting of monitoring the patient and adjusting the dosage and/or timing.

The pharmaceutical compositions can be included in a container, pack, or dispenser together with instructions for administration.

Also provided herein is a conjoint therapy wherein one or more other therapeutic agents are administered with a compound or a pharmaceutical composition comprising a compound provided herein. Such conjoint treatment may be achieved by way of the simultaneous, sequential, or separate dosing of the individual components of the treatment.

### DEFINITIONS

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about”, whether or not the term is explicitly used, unless explicitly stated otherwise. As used herein, the singular forms “a,” “an,” and “the” include plural referents unless the context clearly dictates otherwise.

A “subject,” as used herein, includes both humans and other animals, particularly mammals. Thus, the methods are applicable to both human therapy and veterinary applications. In some embodiments, the patient is a mammal, for example, a primate. In some embodiments, the patient is a human.

A “therapeutically effective” amount of a compound provided herein is typically one which is sufficient to achieve the desired effect and may vary according to the nature and severity of the disease condition, and the potency of the compound. It will be appreciated that different concentrations may be employed for prophylaxis than for treatment of an active disease.

The terms “treatment” and “prevention” are art-recognized and include administration of one or more of the compounds or pharmaceutical compositions provided herein. If it is administered prior to clinical manifestation of the unwanted condition (e.g., disease or other unwanted state of the subject) then the treatment is preventative, (i.e., it protects the subject against developing the unwanted condition). As used in this context, the term “prevent” means to slow or prevent the onset of at least one symptom of a disorder as provided herein. For example, such prevention may be prompted by a likelihood of exposure to an infective agent (e.g., a virus) or when a subject exhibits other symptoms that indicate onset of a disorder (e.g., a metabolic disorder or cardiovascular disorder) may be likely. Alternatively, if it is administered after manifestation of the unwanted condition, the treatment is therapeutic, (i.e., it is intended to diminish, ameliorate, or stabilize the existing unwanted condition or side effects thereof). As used in this context, to “treat” means to ameliorate at least one symptom of a disorder as provided herein.

The term “modulate” as used herein means regulating (e g, inhibiting or activating) the activity of a sirtuin.

The term, “compound,” as used herein is meant to include all stereoisomers, geometric isomers, and tautomers of the structures depicted. Compounds herein identified by name or structure as one particular tautomeric form are intended to include other tautomeric forms unless otherwise specified.

In some embodiments, a compound provided herein, or salt thereof, is substantially isolated. By “substantially isolated” is meant that the compound is at least partially or substantially separated from the environment in which it was formed or detected. Partial separation can include, for example, a composition enriched in the compound provided herein. Substantial separation can include compositions containing at least about 50%, at least about 60%, at least about 70%, at least about 80%, at least about 90%, at least about 95%, at least about 97%, or at least about 99% by weight of the compound provided herein, or salt thereof. Methods for isolating compounds and their salts are routine in the art.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable salt” refers to the relatively non-toxic, inorganic and organic acid addition salts of a compound provided herein. These salts can be prepared in situ during the final isolation and purification of a compound provided herein, or by separately reacting the compound in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, and amino acid salts, and the like. (See, for example, Berge et al. (1977) “Pharmaceutical Salts”, J. Pharm. Sci. 66: 1-19.)

In some embodiments, a compound provided herein may contain one or more acidic functional groups and, thus, is capable of forming pharmaceutically acceptable salts with pharmaceutically acceptable bases. The term “pharmaceutically acceptable salts” in these instances refers to the relatively non-toxic inorganic and organic base addition salts of a compound provided herein. These salts can likewise be prepared in situ during the final isolation and purification of the compound, or by separately reacting the purified compound in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

### EXAMPLES

The invention is further described in the following examples, which do not limit the scope of the invention described in the claims.

### Example 1

### Compound Screen

A publically disclosed, small molecule library consisting of approximately 13,000 compounds was screened using a commercially available (Cayman Chemical) enzyme activity assay. Deacetylase activity of sirtuins SIRT1, SIRT2, SIRT3, and SIRT6 was used as a surrogate to identify compounds with the potential for having broad-range antiviral activity. The compounds were tested at a single concentration (50 μM) in microtiter plates and their activity against the four sirtuins was recorded as a change in fluorescent signal intensity. Z-scores—the number of standard deviations from the plate mean—were calculated and the compounds that significantly (Z-score≦−2 or ≧2) modulated the activity of any of the tested sirtuins individually or in combination were selected for further antiviral analyses. Activation of sirtuin activity generated positive Z-scores and inhibition of sirtuin activity produced negative Z-scores.

### Example 2

### Assessing Antiviral Activity Against Human Cytomegalovirus (HCMV)

The identified compounds were further tested to gauge their antiviral activity. To assess the antiviral activity of these structures, the compounds were tested against human cytomegalovirus (HCMV) in vitro. Human diploid MRC5 fibroblasts were infected with an HCMV AD169 strain at a multiplicity of 1 infectious unit/cell. Two hours following infection, the cells were treated with the compounds at 6 different concentrations (50, 25, 12.5, 6.3, 3.1, and 1.6 μM). DMSO (vehicle) served as a negative control. At 96 hours after infection, the amount of virus released into the medium was quantified by fluorescent focus assay to determine the 50 and 90% inhibitory concentration (IC50 and IC90) of the drugs. The cells were used for the determination of 50% cytotoxic concentration (CC50) using a tetrazolium-based cell viability assay. Under these conditions, the DMSO-treated control wells produced ˜2.0×10⁴ infectious viruses/mL. As shown in Table 1, 28 of the compounds showed antiviral activity against HCMV at IC50 values <50 μM without inducing cytotoxicity in the cells at antiviral concentrations.

TABLE 1

| | | | | | HCMV | HCMV | |
|---|---|---|---|---|---|---|---|
| | | | | | IC50 | IC90 | CC50 |
| Structure | SIRT1 | SIRT2 | SIRT3 | SIRT6 | (μM) | (μM) | (μM) |
| 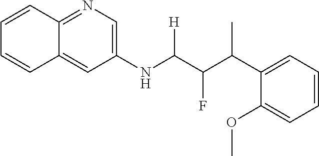 [SMILES: COc1ccccc1C(C)C(F)CNc1cnc2ccccc2c1] | +++ | ++ | na | +++ | 1.19 | 2.24 | >50 |
| 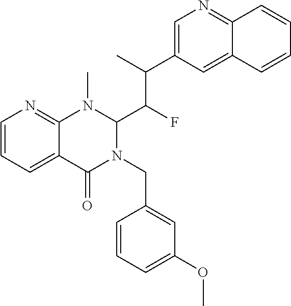 [SMILES: COc1cccc(CN2C(=O)c3cccnc3N(C)C2C(F)C(C)c2cnc3ccccc3c2)c1] | +++ | +++ | +++ | +++ | 1.54 | 15.5 | 25 < CC50 < 50 |
| 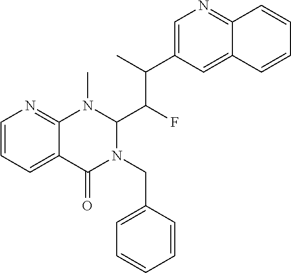 [SMILES: CC(c1cnc2ccccc2c1)C(F)C1N(Cc2ccccc2)C(=O)c2cccnc2N1C] | +++ | +++ | +++ | +++ | 1.74 | 16.13 | >50 |
| 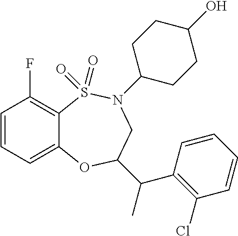 [SMILES: CC(c1ccccc1Cl)C1CN(C2CCC(O)CC2)S(=O)(=O)c2c(F)cccc2O1] | na | na | na | +++ | 2.33 | 31.29 | >50 |
| 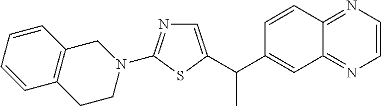 [SMILES: CC(c1ccc2nccnc2c1)c1cnc(N2CCc3ccccc3C2)s1] | −− | −− | na | na | 4.58 | 5.95 | >50 |
| 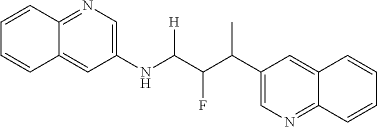 [SMILES: CC(c1cnc2ccccc2c1)C(F)CNc1cnc2ccccc2c1] | na | na | na | +++ | 6.89 | 17.57 | >50 |
| 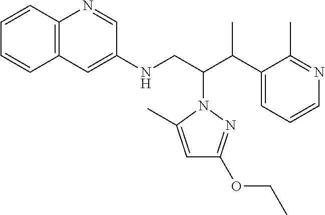 [SMILES: CCOc1cc(C)n(C(CNc2cnc3ccccc3c2)C(C)c2cccnc2C)n1] | ++ | ++ | +++ | +++ | 8.26 | 33.86 | >50 μM |
| 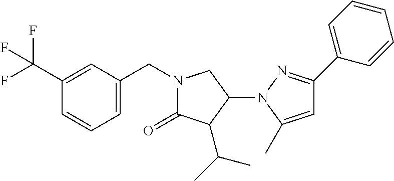 [SMILES: Cc1cc(-c2ccccc2)nn1C1CN(Cc2cccc(C(F)(F)F)c2)C(=O)C1C(C)C] | ++ | na | na | ++ | 8.43 | 18.84 | 25 < CC50 < 50 |
| 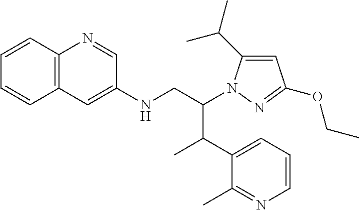 [SMILES: CCOc1cc(C(C)C)n(C(CNc2cnc3ccccc3c2)C(C)c2cccnc2C)n1] | +++ | na | +++ | +++ | 9.32 | 41.3 | >50 |
| 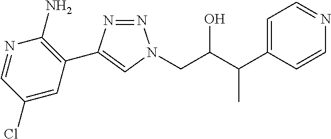 [SMILES: CC(c1ccncc1)C(O)Cn1cc(-c2cc(Cl)cnc2N)nn1] | +++ | na | ++ | +++ | 9.71 | 33.83 | >50 |
| 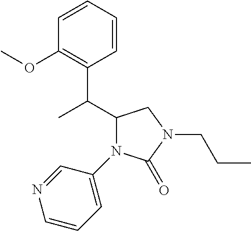 [SMILES: CCCN1CC(C(C)c2ccccc2OC)N(c2cccnc2)C1=O] | na | na | na | +++ | 9.91 | 32.2 | >50 |
| 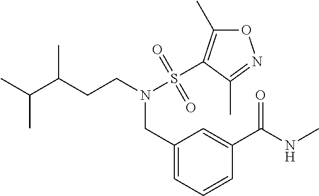 [SMILES: CNC(=O)c1cccc(CN(CCC(C)C(C)C)S(=O)(=O)c2c(C)noc2C)c1] | na | na | na | +++ | 10.15 | 32.79 | 25 < CC50 < 50 |
| 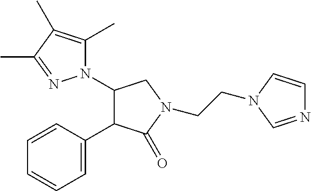 [SMILES: Cc1nn(C2CN(CCn3ccnc3)C(=O)C2c2ccccc2)c(C)c1C] | −−− | −−− | −−− | na | 10.96 | 38.46 | >50 |
| 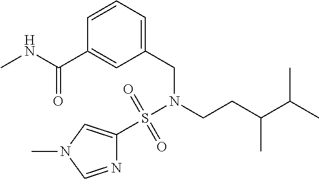 [SMILES: CNC(=O)c1cccc(CN(CCC(C)C(C)C)S(=O)(=O)c2cn(C)cn2)c1] | na | ++ | na | na | 13.39 | 41.78 | >50 |
| 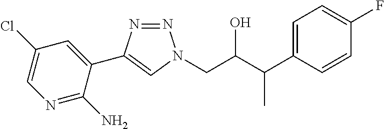 [SMILES: CC(c1ccc(F)cc1)C(O)Cn1cc(-c2cc(Cl)cnc2N)nn1] | +++ | na | na | ++ | 15.06 | 35.55 | >50 |
| 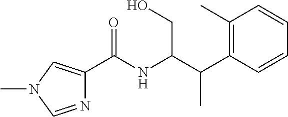 [SMILES: Cc1ccccc1C(C)C(CO)NC(=O)c1cn(C)cn1] | −−− | −−− | na | −− | 15.82 | 41.91 | >50 |
| 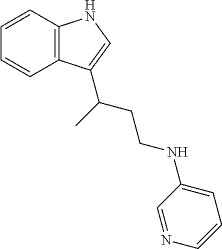 [SMILES: CC(CCNc1cccnc1)c1c[nH]c2ccccc12] | na | +++ | na | na | 15.85 | 43.08 | >50 |
| 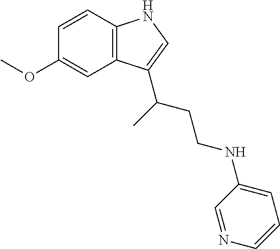 [SMILES: COc1ccc2[nH]cc(C(C)CCNc3cccnc3)c2c1] | na | +++ | na | na | 17.39 | 46.48 | >50 |
| 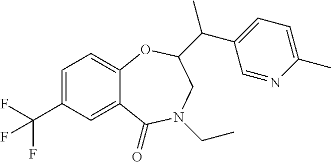 [SMILES: CCN1CC(C(C)c2ccc(C)nc2)Oc2ccc(C(F)(F)F)cc2C1=O] | na | +++ | na | na | 18.43 | 42.32 | >50 |
| 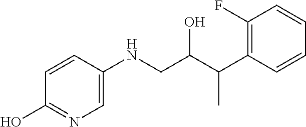 [SMILES: CC(c1ccccc1F)C(O)CNc1ccc(O)nc1] | na | na | +++ | +++ | 19.43 | 43.96 | >50 |
| 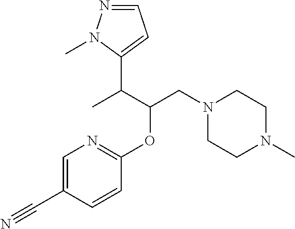 [SMILES: CC(c1ccnn1C)C(CN1CCN(C)CC1)Oc1ccc(C#N)cn1] | −− | na | na | +++ | 21.12 | 42.39 | >50 |
| 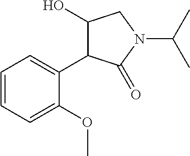 [SMILES: COc1ccccc1C1C(=O)N(C(C)C)CC1O] | na | +++ | na | na | 23.88 | 47.23 | >50 |
| 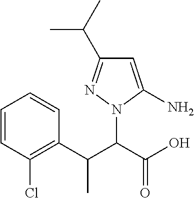 [SMILES: CC(C)c1cc(N)n(C(C(=O)O)C(C)c2ccccc2Cl)n1] | −− | −− | na | na | 25.48 | 57.91 | >50 |
| 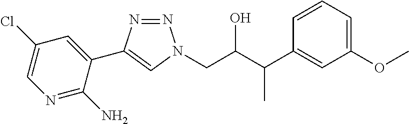 [SMILES: COc1cccc(C(C)C(O)Cn2cc(-c3cc(Cl)cnc3N)nn2)c1] | +++ | na | na | +++ | 26.2 | 44.3 | >50 |
| 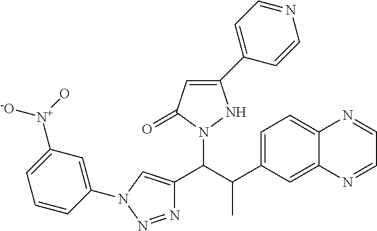 | −− | −− | na | na | 27.44 | 51.76 | >50 |
| 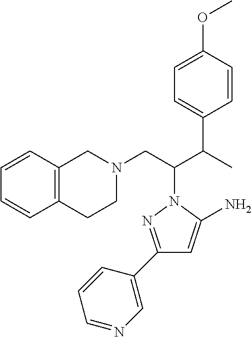 [SMILES: COc1ccc(C(C)C(CN2CCc3ccccc3C2)n2nc(-c3cccnc3)cc2N)cc1] | −−− | −− | na | na | 27.65 | 55.66 | >50 |
| 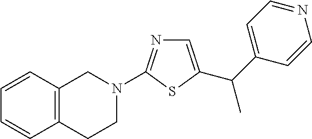 [SMILES: CC(c1ccncc1)c1cnc(N2CCc3ccccc3C2)s1] | na | −−− | na | na | 28.3 | 41.9 | >50 |
| 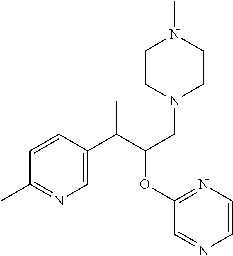 [SMILES: Cc1ccc(C(C)C(CN2CCN(C)CC2)Oc2cnccn2)cn1] | na | na | na | +++ | 30.24 | 56 | >50 |

Plus sign (+) indicates an increase, minus sign (−) indicates a decrease in sirtuin deacetylase activity.
Z-score ≧ 3 = +++, 2 ≦ Z-score < 3 = ++, −2 ≦ Z-score < 2 = no activity (na), −3 < Z-score ≦ −2 = −−, Z-score ≦ −3 = −−−

### Example 3

### Assessing Broad Spectrum Antiviral Activity

Two compounds were selected for further testing:

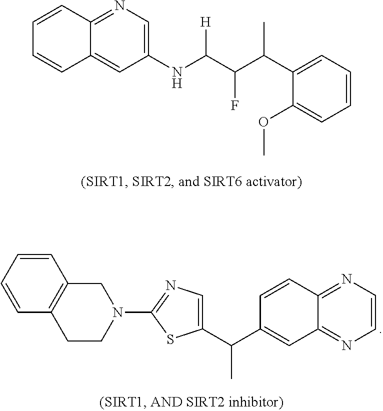 [SMILES: COc1ccccc1C(C)C(F)CNc1cnc2ccccc2c1 ; CC(c1ccc2nccnc2c1)c1cnc(N2CCc3ccccc3C2)s1]

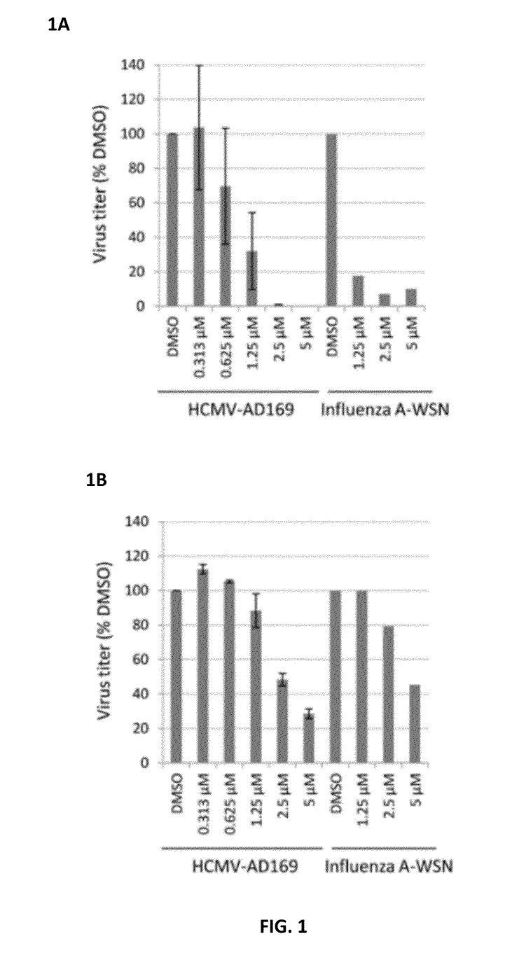

These compounds inhibited HCMV production with the lowest IC50 values among the sirtuin activators and inhibitors described above. The compounds were tested against a second, distinct virus, influenza A in MRC5 cells. Cells were infected with HCMV AD169 or influenza A WSN and treated with the compounds at different concentrations at 2 hours after HCMV and 1 hour after influenza A infection. Virus yield in the culture supernatant was assayed at 96 hours after HCMV and 24 hours after influenza A infection. As shown in FIG. 1, both drugs inhibited the growth of HCMV and influenza A in a dose-dependent manner, indicating that the observed antiviral activities are not virus-specific but rather target multiple and distinct viruses.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating or preventing a viral infection in a subject, the method comprising administering to the subject a therapeutically effective amount of at least one compound selected from the group consisting of: 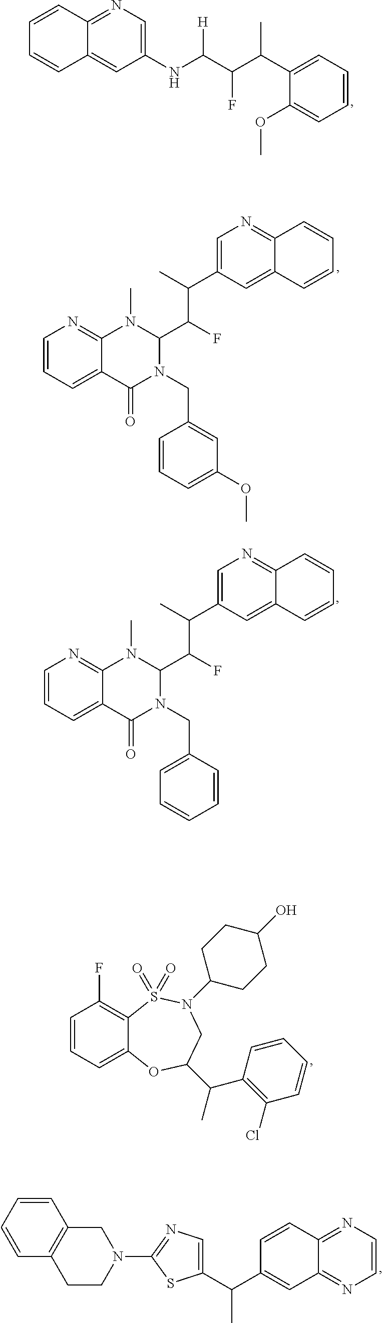 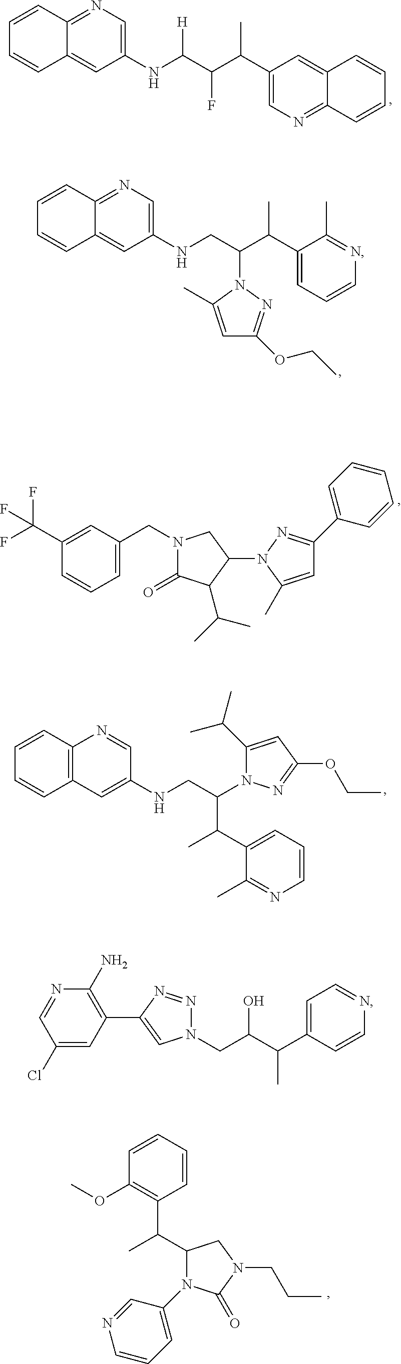 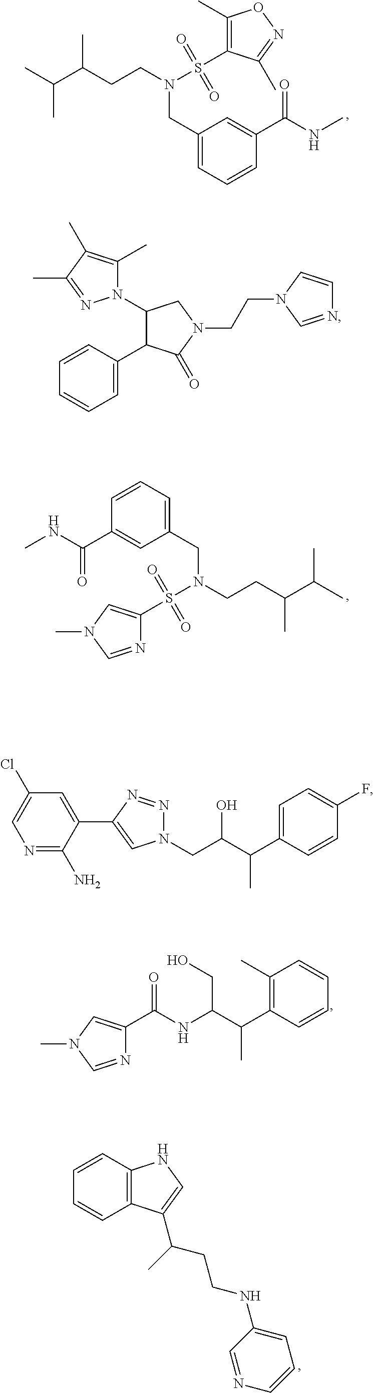 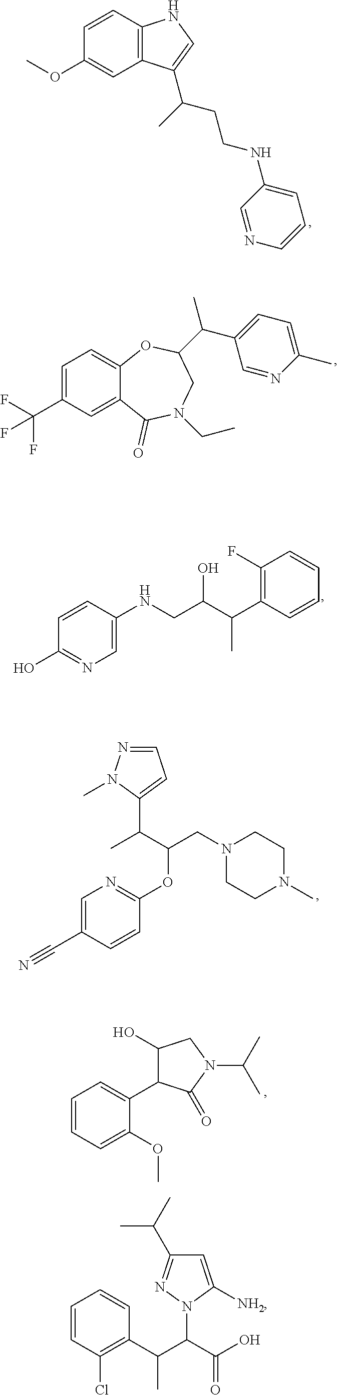 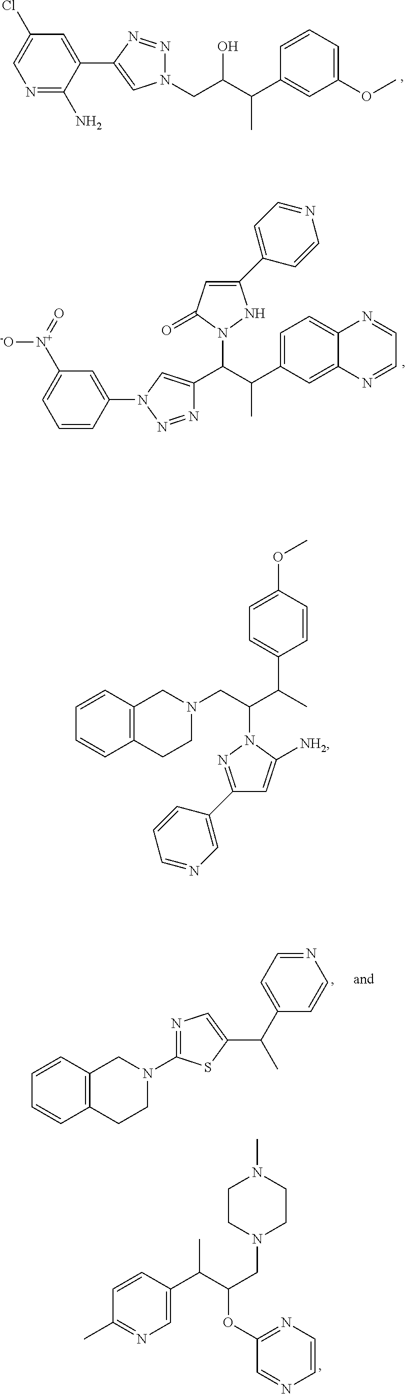
or a pharmaceutically acceptable salt form thereof.

2. A method of inhibiting virus production comprising contacting a virus-infected cell with a therapeutically effective amount of at least one compound selected from the group consisting of: 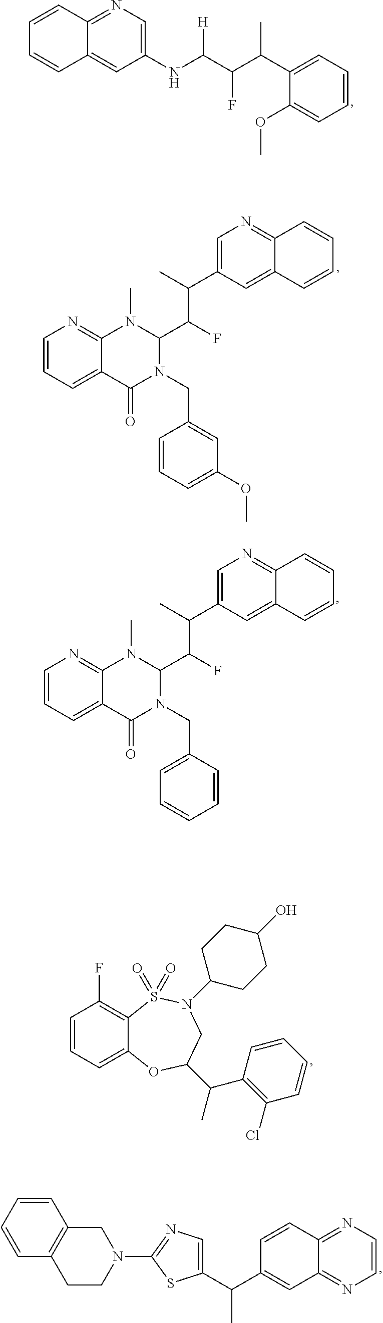 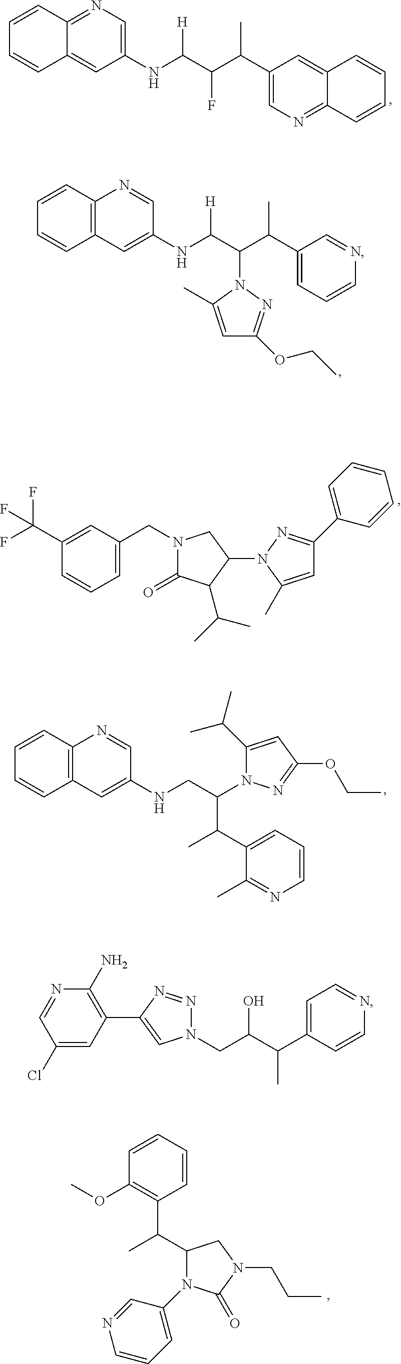 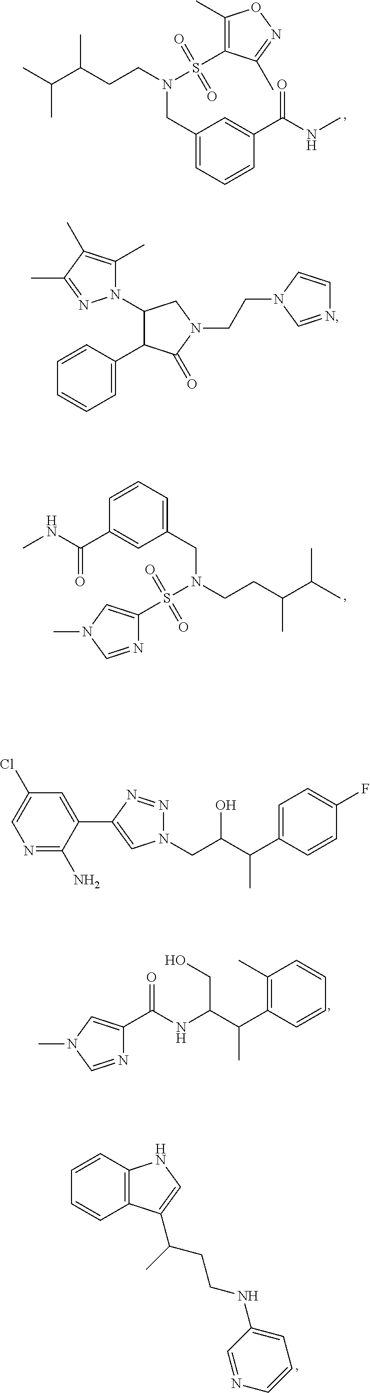 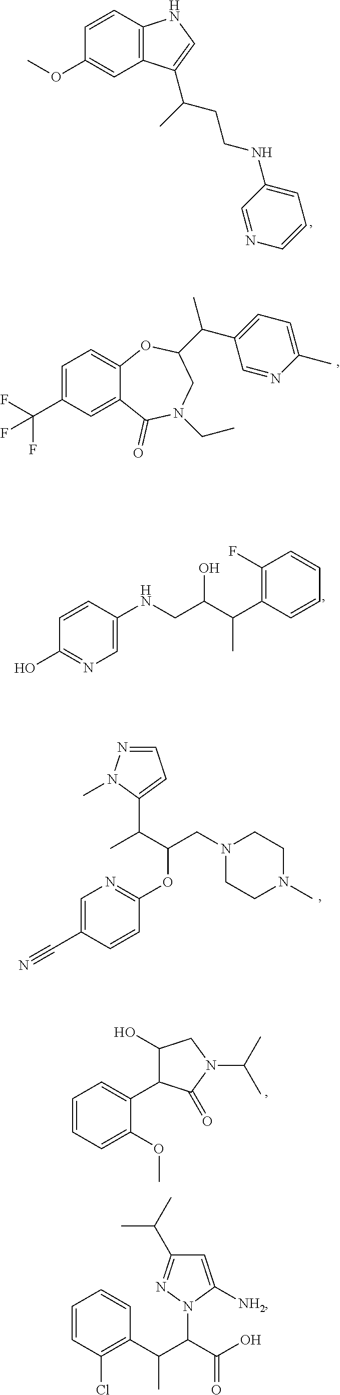 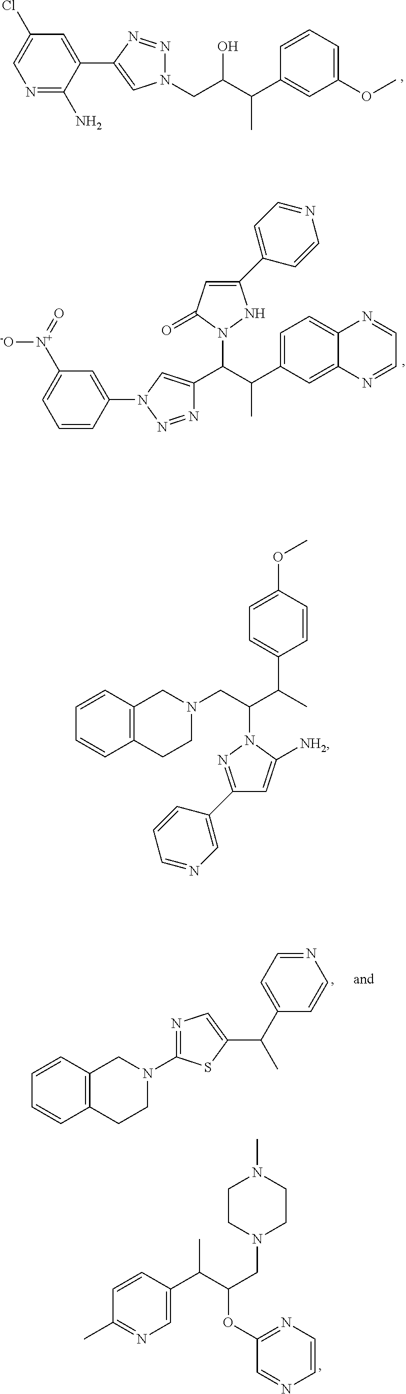
or a pharmaceutically acceptable salt form thereof.

3.-6. (canceled)

7. The method of claim 1, wherein the compound inhibits SIRT1 and SIRT2 and is: 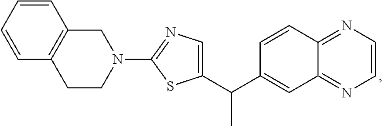 [SMILES: CC(c1ccc2nccnc2c1)c1cnc(N2CCc3ccccc3C2)s1]
or a pharmaceutically acceptable salt thereof.

8.-10. (canceled)

11. The method of claim 1, wherein the compound activates SIRT1, SIRT2 and SIRT6 and is: 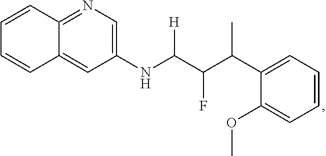 [SMILES: COc1ccccc1C(C)C(F)CNc1cnc2ccccc2c1]
or a pharmaceutically acceptable salt thereof.

12. (canceled)

13. The method of claim 1, wherein the method further comprises administering a therapeutically effective amount of an antiviral agent.

14.-16. (canceled)

17. The method of claim 1, wherein the virus is selected from the group consisting of: HCMV, human papillomavirus, herpes virus, adenovirus, BK virus, JC virus, influenza virus, hepatitis B virus, hepatitis C virus, and HIV.

18.-60. (canceled)

61. A method of modulating one or more sirtuins in a cell, the method comprising contacting a cell with an effective amount of at least one compound selected from the group consisting of: 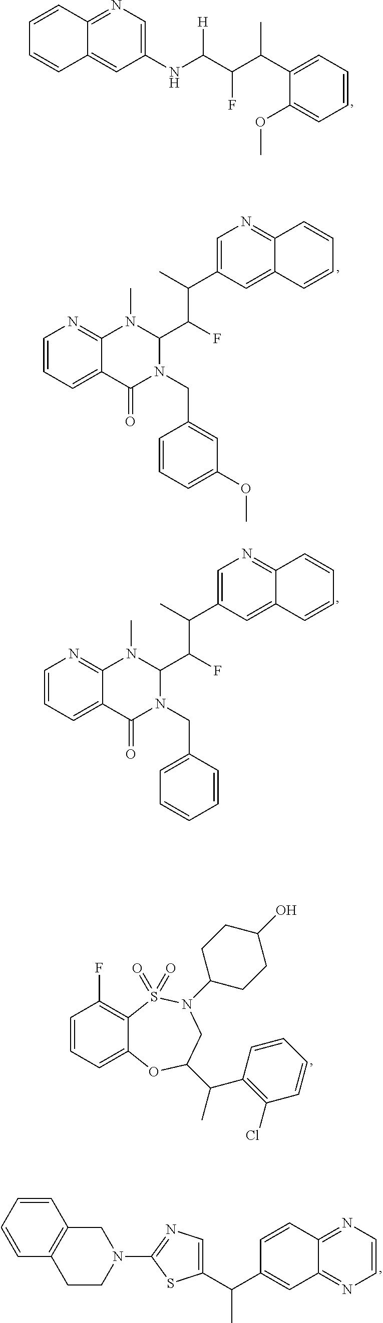 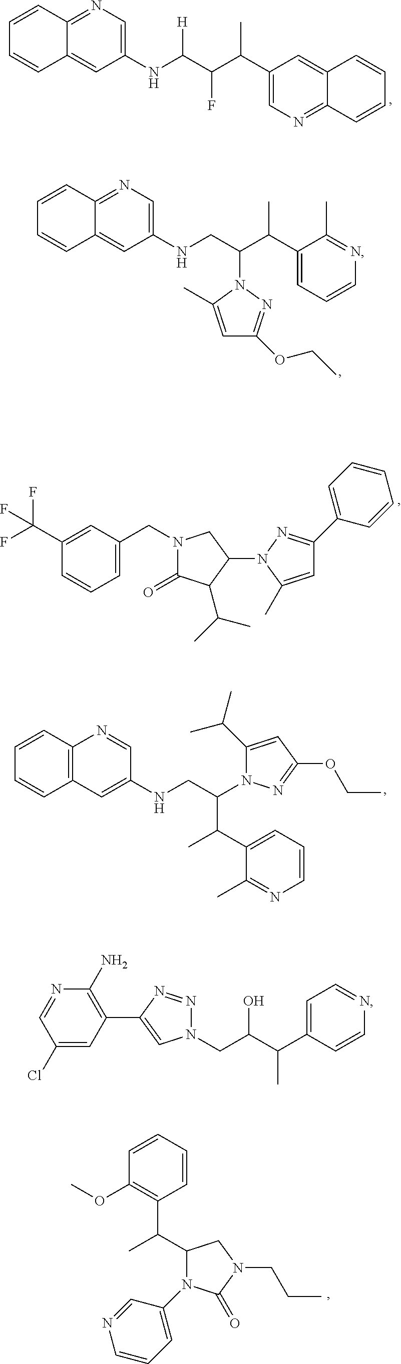 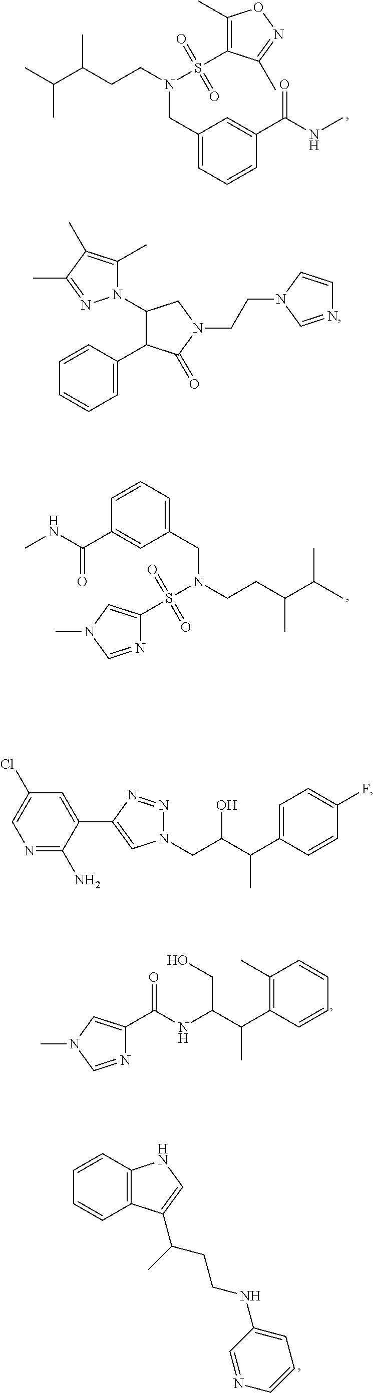 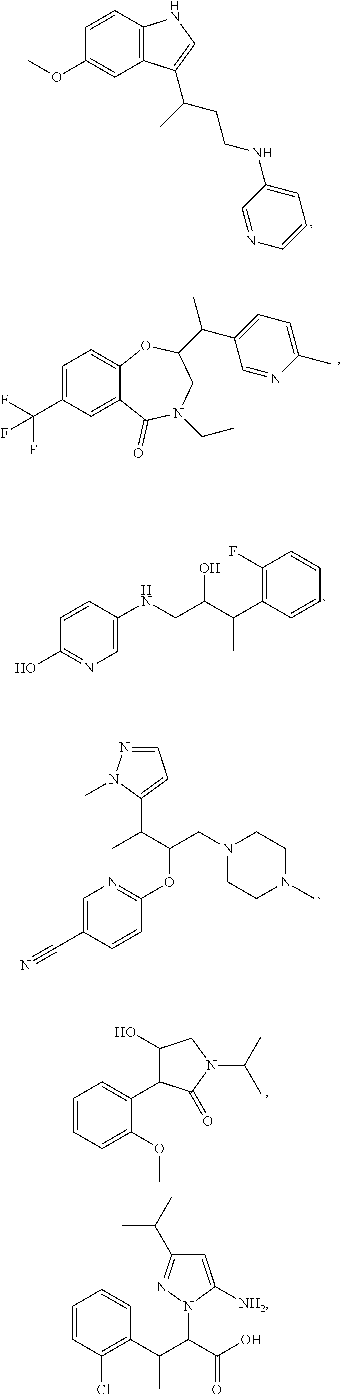 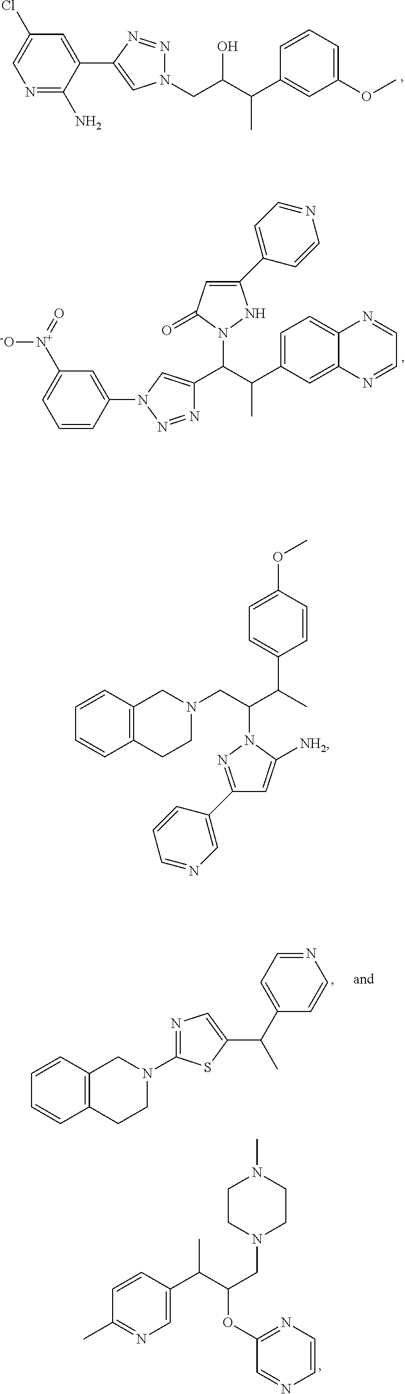
or a pharmaceutically acceptable salt thereof.

62. (canceled)

63. The method of claim 61, wherein the compound inhibits two or more sirtuins selected from the group consisting of: SIRT1, SIRT2, SIRT3, SIRT4, SIRT5, SIRT6, and SIRT7.

64. The method of claim 63, wherein the compound inhibits SIRT1 and SIRT2.

65. The method of claim 64, wherein the compound is: 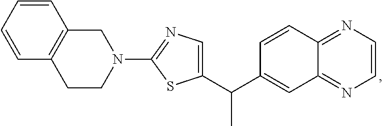 [SMILES: CC(c1ccc2nccnc2c1)c1cnc(N2CCc3ccccc3C2)s1]
or a pharmaceutically acceptable salt thereof.

66. (canceled)

67. The method of claim 61, wherein the compound activates two or more sirtuins selected from the group consisting of: SIRT1, SIRT2, SIRT3, SIRT4, SIRT5, SIRT6, and SIRT7.

68. The method of claim 67, wherein the compound activates two or more sirtuins selected from the group consisting of: SIRT1, SIRT2, and SIRT6.

69. The method of claim 68, wherein the compound is: 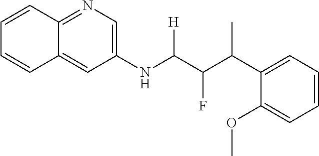 [SMILES: COc1ccccc1C(C)C(F)CNc1cnc2ccccc2c1]
or a pharmaceutically acceptable salt thereof.

70. (canceled)

71. The method of claim 61, wherein the contacting is in vivo.

72. The method of claim 71, wherein the contacting comprises administering the compound to a mammalian subject.

73. (canceled)

74. (canceled)

75. The method of claim 72, wherein the cell is a virus-infected cell.

76. (canceled)

77. (canceled)

78. The method of claim 2, wherein the compound inhibits SIRT1 and SIRT2 and is: 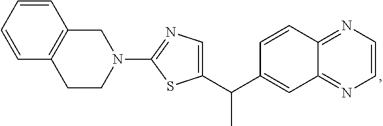 [SMILES: CC(c1ccc2nccnc2c1)c1cnc(N2CCc3ccccc3C2)s1]
or a pharmaceutically acceptable salt thereof.

79. The method of claim 2, wherein the compound activates SIRT1, SIRT2 and SIRT6 and is: 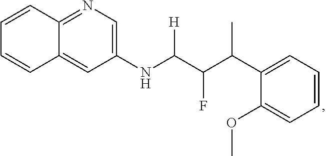 [SMILES: COc1ccccc1C(C)C(F)CNc1cnc2ccccc2c1]
or a pharmaceutically acceptable salt thereof.

80. The method of claim 2, wherein the method further comprises administering a therapeutically effective amount of an antiviral agent.

81. The method of claim 2, wherein the virus is selected from the group consisting of: HCMV, human papillomavirus, herpes virus, adenovirus, BK virus, JC virus, influenza virus, hepatitis B virus, hepatitis C virus, and HIV.
